(19) United States
(12) Patent Application Publication
(10) Pub. No.: US 2026/0130996 A1
(43) Pub. Date: May 14, 2026

(54) ENGINEERED MACROPHAGES FOR USE IN TREATING CANCER

(71) Applicants: Whitehead Institute for Biomedical Research, Cambridge, MA (US); President and Fellows of Harvard College, Cambridge, MA (US)

(72) Inventors: Kipp Weiskopf, Wellesley, MA (US); Andrew Khalil, Cambridge, MA (US); Rudolf Jaenisch, Brookline, MA (US); David J. Mooney, Sudbury, MA (US); Joshua Brockman, Roxbury, MA (US)

(73) Assignees: Whitehead Institute for Biomedical Research, Cambridge, MA (US); President and Fellows of Harvard College, Cambridge, MA (US)

(21) Appl. No.: 19/118,661

(22) PCT Filed: Oct. 6, 2023
(86) PCT No.: PCT/US2023/034666

§ 371 Date: Apr. 4, 2025

Related U.S. Application Data
(60) Provisional application No. 63/414,352, filed on Oct. 7, 2022.

(51) Int. Cl. A61K 40/17; A61K 39/395; A61K 40/31; A61K 40/42; A61P 35/00; G01N 33/50

(52) U.S. Cl. CPC A61K 40/17; A61K 39/3955; A61K 40/31; A61K 40/4211; A61P 35/00; G01N 33/5011

(57) ABSTRACT

The present disclosure relates to pharmaceutical compositions comprising engineered macrophages and methods of use thereof. In some aspects, the present disclosure relates to engineered macrophages derived from pluripotent stem cells. In some embodiments, the stem cell genome is edited to knock-out (e g., SIRPa) and/or knock-in (e g., CAR) genes of interest prior to differentiation into macrophages. In some embodiments, engineered macrophages lack a SIRPa receptor. In some embodiments, the engineered macrophages express chimeric antigen receptors that induce and/or enhance phagocytosis. Alternatively, or additionally, in some embodiments, macrophages are engineered with combinatorial antigen-sensing circuits to improve tumor recognition and phagocytosis. Other aspects of the disclosure relate to methods of treating cancer in a subject and/or methods of enhancing cancer cell phagocytosis.

### RELATED APPLICATIONS

This application claims priority under 35 U.S.C. § 119(e) to U.S. Provisional Patent Application Ser. No. 63/414,352, filed Oct. 7, 2022, entitled “ENGINEERED MACROPHAGES FOR USE IN TREATING CANCER,” by K. Weiskopf et al., which is incorporated herein by reference in its entirety.

### FIELD

The present disclosure generally relates to engineered macrophages and their uses.

### BACKGROUND

Macrophages have shown promise as cancer immunotherapies in their ability to phagocytose solid tumor cells. This process is however limited by the inhibition of phagocytosis through the CD47-SIRPa signaling axis. Inhibition of this axis via CD47-opsonizing antibodies has been shown to increase phagocytosis of cancer cells with upregulated CD47. However, requisite use of these antibodies and reliance on primary sources of human macrophages are limiting. Thus, improvements are needed.

### SUMMARY

Macrophages are large phagocytotic cells which primarily act to defend the host against infection, injury, and cancer metastasis. During organogenesis, macrophages derived from the embryonic yolk sac and fetal liver precursors are seeded throughout tissues, persisting in the adult as resident self-maintaining populations, which turn over locally under steady state conditions. After birth, bone marrow-derived blood monocytes replenish resident macrophage populations with high turnover. Thus, human monocytes obtained directly from either blood and/or bone marrow represent the primary source of macrophages for therapeutic purposes. Alternatively, or additionally, macrophages may be obtained directly from body cavity lavages (e.g., alveolar, peritoneal, etc.). However, these collection methods typically result in low yields and produce macrophages with heterogenous phenotypes. Additionally, monocyte-differentiated macrophages typically fail to proliferate as they differentiate in vitro and are difficult to genetically modify, making cell line generation and genetic engineering infeasible.

Thus, aspects of the disclosure relate to producing macrophages from alternative cell sources. The inventors of the disclosure have found that macrophages derived from pluripotent stem cells (e.g., embryonic stem(ES) cells or induced pluripotent stem cells, (iPSCs), etc.) have several advantages over monocyte-derived macrophages. For example, induced pluripotent stem cells have the advantage of being easily obtained from adult somatic cells, have a high self-renewal capacity, and various differentiation protocols result in macrophages with similar phenotypes. Further, the differentiation of macrophages from iPSCs potentially allows macrophages to be obtained from any individual, of any genetic background, in unlimited quantities and permits the standardization of macrophage populations. Additionally, iPSC-derived macrophages can be genetically edited, are scalable, and have greater clinical applicability than their monocyte-derived counterparts.

Aspects of the disclosure also relate to macrophages with improved cell recognition (e.g., specificity) and/or phagocytotic activity by engineering the cell genome to eliminate expression of one or more genes (e.g., SIRPa) and/or increase expression of one or more CARs (e.g., CAR-P). In some embodiments, the eliminated gene product is expressed on the cell surface. The mechanisms by which macrophages phagocytize foreign material is known in the art. Without wishing to be bound by any particular theory, professional phagocytes, such as macrophages, express several phagocytic receptors that activate signaling pathways resulting in phagocytosis (e.g., Fc receptors and complement receptors). For example, antibodies comprise a Fab domain configured to bind to a target antigen, and a Fc domain configured to bind to an immunoglobin Fc receptor on the of a macrophage. Binding of the Fc domain to its conjugate receptor triggers the macrophage to engulf and digest the target (e.g., cancer cell) bearing the antigen.

Cell expression of CD47 is a common mechanism by which cells protect themselves from phagocytosis. For example, CD47 expression is required to protect transfused red blood cells, platelets, and lymphocytes from rapid elimination by splenic macrophages. Mobilized hematopoietic stem cells protect themselves from phagocytosis by increasing CD47 expression as they pass through phagocyte-lined sinusoids and decrease CD47 expression after relocating to marrow niches.

Pathogens and tumor cells also leverage CD47 to gain immune privilege. For example, CD47 is expressed on many human solid tumors, including ovarian, breast, colon, bladder, glioblastoma, hepatocellular carcinoma, and prostate tumors. Without wishing to be bound by any particular theory, it is believed that cancer cells express CD47 to avoid phagocytosis by binding to signal regulatory protein-alpha (SIPRa) expressed on the macrophages and dendritic cells. Upon binding CD47, SIRPa initiates a “do not eat me” signaling cascade that results in the inhibition of phagocytosis. Current approaches to inhibiting this signaling axis rely on CD47 antibodies that bind to the SIRPa receptor and physically block binding to CD47 ligands on the surface of cancer cells.

As such, aspects of the present disclosure generally relate to engineered macrophages with improved phagocytotic activity and cell recognition capabilities, for example, by inhibiting the CD47-SIRPa signaling axis. The present disclosure stems from the discovery that pluripotent stem cell-derived (PSC) macrophages engineered without a SIRPa gene exhibit enhanced phagocytosis in the absence of CD47 neutralizing antibodies. Opsonization using any IgG serotype is sufficient to stimulate phagocytosis by said engineered macrophages, which is significant because IgGs represent approximately 75% of serum antibodies in humans and are the most common type of antibody found in the blood circulation. In other words, IgG antibodies replace CD47 as the opsin, rendering the engineered macrophages disclosed herein deaf to the CD47 “do not eat me” signal leveraged by human tumor cells.

Thus, in some aspects, the present disclosure relates to engineered immune cells (e.g., macrophages) and/or pharmaceutical compositions comprising said cells with improved phagocytotic activity and cell recognition capabilities derived from pluripotent stem cells (e.g., induced pluripotent stem cells, iPSC cells; true embryonic stem cells, ES cells; by somatic cell nuclear transfer cells, ntES cells; and parthenogenetic embryonic stem cells, pES cells). In some embodiments, the genome of the parent stem cell is edited, for example, using CRISPR/Cas9, to knock-in or knock-out (e.g., SIRPa) a gene of interest. In some embodiments, the SIRPa gene in the parent stem cell is knocked out using CRISPR/Cas9. In some embodiments, the edited parent stem cell is subsequently differentiated into a macrophage.

In some embodiments, the present disclosure relates to an engineered immune cell or population of engineered immune cells comprising a genome that has been edited to eliminate expression of SIRPa (e.g., surface expression) in the cell or population of cells, wherein the engineered immune cell is derived from a pluripotent stem cell, or a primary cell, or a non-stem cell derived immune cell In some embodiments, the primary cell is a macrophage. In some embodiments, the present disclosure relates to an engineered immune cell or population of engineered immune cells comprising a genome that has been edited to eliminate surface expression of SIRPa (e.g., surface expression) in the cell or population of cells.

Additional aspects of the disclosure relate to engineered macrophages with increased tumor specificity and phagocytosis. In some embodiments, increased macrophage tumor specificity is achieved by knocking-in genes encoding chimeric antigen receptors for phagocytosis (CAR-P). Without wishing to be bound by any particular theory, it is believed that the expression of CAR-P constructs by macrophages may increase targeting of tumor cells while simultaneously stimulating the “eat me” signal to initiate phagocytosis. Like their CAR-T cell counterparts, CAR-Ps comprise a target binding extracellular domain (e.g., scFv domain), followed by a hinge sequence, a transmembrane domain, and an intracellular signaling domain. The CAR-Ps impart macrophages with the ability to recognize tumor cells via their scFv region and to trigger phagocytosis of the tumor cell (via the intracellular signaling domain). This occurs at higher specificity and efficacy, relative to endogenous macrophages, due to the CAR construct conferring increased tumor recognition capability to the macrophage.

In some embodiments, the disclosure relates to pharmaceutical composition comprising an engineered immune cell or population of engineered immune cells comprising a genome that has been edited to express chimeric antigen receptors that trigger phagocytosis in the cell or population of cells, and an antibody (e.g. CD47 or IgG antibody). In some embodiments, the engineered immune cell is derived from a pluripotent stem cell or a primary cell, or a non-stem cell derived immune cell, and the genome is edited at a safe harbor locus site.

In some embodiments, increased tumor specificity and phagocytosis is accomplished by engineering macrophages to phagocytize tumor cells bearing multiple antigens. Without wishing to be bound by any particular theory, the design of such macrophages is similar to AND logic gates commonly found in computer programs. For example, some embodiments are directed toward a combinatorically activated macrophage circuit in which a synthetic Notch receptor for one antigen induces the expression of a CAR-P receptor for a second antigen. Binding of the newly expressed CAR-P receptor to its antigen (if present on the tumor cell) triggers the macrophage to phagocytize the tumor cell. Thus, dual-receptor AND-gate macrophages are only armed and activated in the presence of tumor cells bearing two antigens.

In some embodiments, an engineered immune cell or population of engineered immune cells comprises a synthetic Notch (synNotch) receptor comprising an extracellular recognition domain configured to bind to a first antigen; and an inducible chimeric antigen receptor (CAR) configured to bind to a second antigen wherein binding of the synthetic Notch receptor to the first antigen drives expression of the inducible chimeric antigen receptors, and wherein the simultaneous binding of the inducible chimeric antigen receptor to the second antigen stimulates immune cell activation (e.g., phagocytosis).

In some embodiments, an engineered immune cell or population of engineered immune cells comprise a synNotch receptor configured to stimulate cytokine secretion upon binding of the synNotch's extracellular recognition domain of the target antigen. The cytokine secretion profile can be tailored, for example, to promote extravasation and homing to antigen-rich micro-environments.

In some embodiments, an engineered immune cell comprises a genome that has been edited to eliminate expression of one or more target genes, wherein the engineered immune cell is an immune cell derived from a pluripotent stem cell or population of stem cells or a primary cell, or a non-stem cell based immune cell.

Additional aspects of the current disclosure relate to combinations of pharmaceutical compositions and/or combinations of engineered immune cells. For example, in some embodiments, the combination of engineered immune cells comprises an engineered immune cell or population of engineered immune cells comprising a genome that has been edited to (a) eliminate expression of SIRPa in the cell or population of cells, and (b) increase expression of chimeric antigen receptors that triggers said engineered immune cells to phagocytose the cells comprising the antigen.

Alternatively, or additionally, an engineered immune cell or population of engineered immune cells, according to some embodiments, comprises a genome that has been edited to (a) eliminate expression of SIRPa in the cell or population of cells, (b) allow expression of a synthetic Notch receptor comprising an extracellular recognition domain configured to bind to a first antigen, and (c) allow expression of an inducible chimeric antigen receptor configured to bind to second antigen, wherein binding of the synthetic Notch receptor to the first antigen drives expression of the inducible chimeric antigen receptors, and wherein binding of the inducible chimeric antigen receptor to the second antigen stimulates immune cell activation.

In some embodiments, an engineered immune cell or population of engineered immune cells, comprises at least one synthetic Notch receptor comprising an extracellular recognition domain configured to bind to at least one first antigen and at least one inducible chimeric antigen receptor (CAR) configured to bind to at least one second antigen. In some cases, the binding of the at least one synthetic Notch receptor to the at least one first antigen drives expression of the at least one inducible chimeric antigen receptor. In some embodiments, the binding of the at least one inducible chimeric antigen receptor to the at least one second antigen stimulates secretion of one or more cytokines.

Pharmaceutical compositions are also provided. For example, in some embodiments, the pharmaceutical compositions comprises an antibody, an antibody derivative, and/or a fragment thereof and one or more of the engineered immune cells disclosed herein. In some embodiments, a pharmaceutical composition comprises an IgG antibody, an IgG derivative, and/or fragment thereof and one or more of the engineered immune cells disclosed herein.

In some embodiments, a pharmaceutical composition comprises a CD47 antibody, a CD47 antibody derivative, and/or a fragment thereof and one or more of the engineered immune cells disclosed herein.

In some embodiments, a pharmaceutical composition comprises a CD47 antibody, a CD47 antibody derivative, and/or fragment thereof, an IgG antibody, an IgG antibody derivative, and/or fragment thereof and one or more of the engineered immune cells disclosed herein.

Methods of use are also provided. For example, in some embodiments, the method comprises treating cancer in a subject in need thereof, the method comprising administering one or more of the cells, populations of cells, or pharmaceutical compositions described herein. In some embodiments, the method relates to a method of enhancing the phagocytosis of cancer cells, the method comprising administering one or more of the pharmaceutical compositions described herein. Other methods relate to methods of engineering an immune cell with increased anti-tumor activity.

Other aspects of the disclosure are directed toward methods for screening libraries of genes that enhance the function of macrophages (e.g., increase phagocytotic ability). For example, in some embodiments, a method for screening for engineered immune cells with enhanced phagocytotic activity, the method comprising:
(a) inducing gene knockout after differentiating PSC into macrophages using sgRNA libraries;
(b) enriching for phagocytic cells using fluorescence activated cell sorting;
(c) identifying the gene knocked out using traditional sequencing techniques; and
(d) creating a plurality of engineered immune cells, wherein the genome of the engineered immune cell has an edited genome.

### Definitions

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like, Salts derived from appropriate bases include alkali metal, alkaline earth metal, ammonium and N⁺(C₁₋₄ alkyl)4⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2 H₂O) and hexahydrates (R·6 H₂O)).

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers.”

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “inhibits” or “inhibition” in the context of modulating level (e.g., expression and/or activity) of a target (e.g., SIRPa) is not limited to only total inhibition.

Thus, in some embodiments, partial inhibition or relative reduction is included within the scope of the term “inhibition.” In some embodiments, the term refers to a reduction of the level (e.g., expression, and/or activity) of a target (e.g., SIRPa) to a level that is reproducibly and/or statistically significantly lower than an initial or other appropriate reference level, which may, for example, be a baseline level of a target. In some embodiments, the term refers to a reduction of the level (e.g., expression and/or activity) of a target to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of a target.

As used herein, the term “inhibits” or “inhibition” refers to an agent whose presence or level correlates with decreased level or activity of a target to be modulated. In some embodiments, an inhibitor may act directly (in which case it exerts its influence directly upon its target, for example by binding to the target); in some embodiments, an inhibitor may act indirectly (in which case it exerts its influence by interacting with and/or otherwise altering a regulator of a target, so that level and/or activity of the target is reduced). In some embodiments, an inhibitor is one whose presence or level correlates with a target level or activity that is reduced relative to a particular reference level or activity (e.g., that observed under appropriate reference conditions, such as presence of a known inhibitor, or absence of the inhibitor as disclosed herein, etc.).

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. A “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The terms “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount effective to prevent a condition, or one or more symptoms associated with the condition and/or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites.

Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, ²5th ed. ; Hensyl ed.; Williams & Wilkins: Philadelphia, 1990. Exemplary cancers include, but are not limited to, hematological malignancies. The term “hematological malignancy” refers to tumors that affect blood, bone marrow, and/or lymph nodes. Exemplary hematological malignancies include, but are not limited to, leukemia, such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)), lymphoma, such as Hodgkin lymphoma (HL) (e.g., B-cell HL, T-cell HL) and non-Hodgkin lymphoma (NHL) (e.g., B-cell NHL, such as diffuse large cell lymphoma (DLCL) (e.g., diffuse large B-cell lymphoma (DLBCL, e.g., activated B-cell (ABC) DLBCL (ABC-DLBCL))), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt lymphoma, Waldenström's macroglobulinemia (WM, lymphoplasmacytic lymphoma), hairy cell leukemia (HCL), immunoblastic large cell lymphoma, precursor B-lymphoblastic lymphoma, central nervous system (CNS) lymphoma (e.g., primary CNS lymphoma and secondary CNS lymphoma), and T-cell NHL, such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; and multiple myeloma (MM). Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms'tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma), bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma), esophageal cancer (e.g., adenocarcinoma of the esophagus, Barrett's adenocarcinoma); Ewing's sarcoma; ocular cancer (e.g., intraocular melanoma, retinoblastoma), familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “immunotherapy” refers to a treatment of disease by inducing, enhancing, or suppressing an immune response. Immunotherapies designed to elicit or amplify an immune response are classified as activation immunotherapies, while immunotherapies that reduce or suppress an immune response are classified as suppression immunotherapies. Immunotherapy may encompass treatment with a molecular entity (e.g., immunotherapeutic agent) and/or a non-molecular entity (e.g., adoptive cell transfer).

The term “macrophage-directed immunotherapy” refers to a type of cell therapy in which macrophages are used to recognize tumor cells and either (1) recruit other immune cells to the tumor site or (2) to kill the tumor cells by phagocytosis.

The term “immunotherapeutic agent” refers to a molecular entity that induces, enhances, or suppresses an immune response. Immunotherapeutic agents include, but are not limited to, monoclonal antibodies, cytokines, chemokines, vaccines, small molecule inhibitors, and small molecule agonists.

The term “immune checkpoint inhibitor” refers to an agent that blocks certain proteins made by some types of immune system cells (e.g., T cells, macrophages) and some cancer cells. These proteins function to keep immune responses in check and can also function to keep immune system cells (e.g., T cells, macrophages) from killing cancer cells. When these proteins are blocked, immune system function is restored, and the immune system is released enabling the desired immune system cells to kill cancer cells. Some immune checkpoint inhibitors are useful in treating cancer. A “macrophage immune checkpoint inhibitor” functions to stimulate macrophage phagocytosis of cancer cells. For example, CD47 is associated with a macrophage immune checkpoint (CD47/SIRPα as described herein). CD47-blocking therapies thus stimulate macrophage phagocytosis of cancer cells and are effective in treating cancer.

The terms “biologic,” “biologic drug,” and “biological product” refer to a wide range of products, such as vaccines, blood and blood components, allergenics, somatic cells, gene therapy, tissues, nucleic acids, and proteins. Biologics may include sugars, proteins, or nucleic acids, or complex combinations of these substances, or may be living entities such as cells and tissues. Biologics may be isolated from a variety of natural sources (e.g., human, animal, microorganism) and/or may be produced by biotechnological methods and/or other technologies.

The term “antibody” refers to a functional component of serum and is often referred to either as a collection of molecules (antibodies or immunoglobulins) or as one molecule (the antibody molecule or immunoglobulin molecule). An antibody is capable of binding to or reacting with a specific antigenic determinant (the antigen or the antigenic epitope), which in turn may lead to induction of immunological effector mechanisms. An individual antibody is usually regarded as monospecific, and a composition of antibodies may be monoclonal (i.e., consisting of identical antibody molecules) or polyclonal (i.e., consisting of two or more different antibodies reacting with the same or different epitopes on the same antigen or even on distinct, different antigens). Each antibody has a unique structure that enables it to bind specifically to its corresponding antigen, and all natural antibodies have the same overall basic structure of two identical light chains and two identical heavy chains. Antibodies are also known collectively as immunoglobulins. An antibody may be of human or non-human (for example, rodent such as murine, dog, camel, etc.) origin (e.g., may have a sequence originally developed in a human or non-human cell or organism), or may be or comprise a chimeric, humanized, reshaped, or reformatted antibody based, e.g., on a such a human or non-human antibody (or, in some embodiments, on an antigen-binding portion thereof).

In some embodiments, as will be clear from the context, the term “antibody” as used herein encompasses formats that include epitope-binding sequences of an antibody, which such formats include, for example chimeric and/or single chain antibodies (e.g., a nanobody or Fcab), as well as binding fragments of antibodies, such as Fab, Fv fragments or single chain Fv (scFv) fragments, as well as multimeric forms such as dimeric IgA molecules or pentavalent IgM molecules. Also included are bispecific antibodies, bispecific T cell engagers (BiTEs), immune mobilixing monoclonal T cell receptors against cancer (ImmTACs), dual-affinity re-targeting (DART); alternative scaffolds or antibody mimetics (e.g., anticalins, FN3 monobodies, DARPins, Affibodies, Affilins, Affimers, Affitins, Alphabodies, Avimers, Fynomers, Im7, VLR, VNAR, Trimab, CrossMab, Trident); nanobodies, binanobodies, F(ab′)2, Fab′, di-sdFv, single domain antibodies, trifunctional antibodies, diabodies, and minibodies.

The term “therapeutic agent” refers to an agent having one or more therapeutic properties that produce a desired, usually beneficial, effect. For example, a therapeutic agent may treat, ameliorate, and/or prevent disease. In some embodiments, a therapeutic agent may be or comprise a biologic, a small molecule, or a combination thereof.

The term “chemotherapeutic agent” refers to a therapeutic agent known to be of use in chemotherapy for cancer.

The term “targeted agent” refers to an anticancer agent that blocks the growth and spread of cancer by interfering with specific protein“ (”molecular targ”ts”) that are involved in the growth, progression, and spread of cancer. Targeted agents are sometimes called “targeted therapies,” “targeted cancer therapies,” “molecularly targeted drugs,” “molecularly targeted therapies,” or “precision medicines.” Targeted agents differ from standard chemotherapy in that targeted agents act on specific molecular targets that are associated with cancer, whereas many chemotherapeutic agents act on all rapidly dividing cells (e.g., whether or not the cells are cancerous). Targeted agents are deliberately chosen or designed to interact with their target, whereas many standard chemotherapies are identified because they may indiscriminantly kill cells.

The term “tumor antigen” or “tumor associated antigen (TAA)” refers to an antigenic substance produced in tumor cells. In general, antigenic refers to the ability of an antigen to stimulate an immune response in the subject. Tumor antigens may be used as tumor biomarkers to identify cancerous cells.

The term, “PSC-Macs” refers to a macrophage derived from a pluripotent stem cell. The term, “PSC-MAC-SIRPA-KO”, refers to a macrophage derived from a pluripotent stem cell whose genome that has been edited to eliminate expression of the SIRPA gene. The term “PSC-MAC-CAR” refers to a macrophage derived from a pluripotent stem cell whose genome has been edited to induce expression of a CAR-P construct. The term, “PSC-Mac-CAR (tandem)” refers to a macrophage derived from a pluripotent stem cell whose genome has been edited to induce expression of a CAR-P construct wherein the intracellular signaling domain of the CAR comprises two or more domains linked in tandem, for example, to increase the frequency of whole cell engulfment (e.g., a FcRγ domain fused to a CD19 cytoplasmic domain). The term, “PSC-Mac-SIRPa-CAR cells” refers to a macrophage derived from a pluripotent stem cell whose genome has been edited to delete expression of SIRPA and to induce expression of a CARP. The term, “PSC-Mac-DUAL” refers to a macrophage derived from a pluripotent stem cell whose genome has been edited to include a synNotch signaling circuit coupled to a CAR-P signaling circuit.

The term, “pluripotent” refers to cells that are able to self-renew by dividing and developing into the three primary groups of cells that make up the human body, including the ectoderm (e.g., giving rise to the skin and nervous system), endoderm (e.g., forming the gastrointestinal and respiratory tracts, endocrine glands, liver, and pancreas), and mesoderm (e.g., forming bone, cartilage, most of circulatory system, muscles, connective tissue).

The ter”, “true embryonic stem cells” refers to pluripotent stem cells derived from the inner cell mass of a blastocyst. The term, “induced pluripotent stem cells (iPSCs)” refers to a type of pluripotent stem cell that can be generated directly from somatic cells. The term, “somatic cell nuclear transfer cells (ntES cells)” refers to the cells of an inner cell mass of a blastocyst obtained from a laboratory made embryo generated by combining an enucleated oocyte (e.g., egg cell) and implanting a donor nucleus from a somatic cell. The term, “parthenogenetic embryonic stem cells (pES cells)” refers to cells derived from the inner cell mass of blastocysts obtained from unfertilized oocytes that have been activated using parthenogenesis (e.g., asexual reproduction)

### BRIEF DESCRIPTION OF THE DRAWINGS

The accompanying drawings, which are incorporated in and constitute a part of this specification, illustrate several embodiments of the disclosure and together with the description, provide non-limiting examples of the disclosure.

FIGS. 1A-1B are exemplary schematics showing the SIRPa signaling axis in macrophage cancer phagocytosis.

FIG. 2 shows an experimental schematic illustrating the evaluation of macrophages derived from human embryonic stem cells (H1/WA01). Y-axis shows growth of cancer cells as GFP+ area. Increasing values on the Y-axis indicate cancer cell growth while decreases show cancer cell phagocytosis/inhibition. Results show PSC-macrophages are able to phagocytosis whole solid tumor cancer cells in a dose-dependent manner with respect to macrophage number and the inclusion of CD47-opsonizing antibody. This antibody inhibits SIRPa on the PSC-derived macrophage and provides opsonization to allow cancer cell phagocytosis.

FIGS. 3A-3B show that PSCs can be engineered via CRISPR/Cas9 target gene insertion and deletion to provide engineered macrophages with knock-in and knock-out function. Here, H1 (WA01) hESCs was targeted at the AAVS1 safe harbor locus to insert/nock-in constitute expression a reporter fluorescent protein (H1-GFP). Expression of SIRPa in the stem cells was also knocked out, and macrophages were derived.

FIG. 4 shows an experimental schematic illustrating the evaluation of the function of engineered KO PSC-macrophages.

FIG. 5 shows that PSC-Mac-SIRPa demonstrate some base line phagocytosis against DLD-1 adenocarcinoma in the absence of CD47 antibody. This shows that removing the SIRPa signaling pathway is not fully sufficient for promoting cancer cell phagocytosis, according to some embodiments.

FIG. 6 shows non-limiting, representative images of the assay shown in FIG. 5 for wildtype (WT) and PSC-Mac-SIRPa KO #19. Puncta show areas of cancer cell phagocytosis.

FIG. 7 shows KO of SIRPa in engineered PSC-macrophages dramatically increases cancer cell phagocytosis with inclusion of CD47 opsonizing antibody. This suggests that SIRPa KO imparts functionality, but that opsonization is still required for phagocytosis.

FIG. 8 shows non-limiting, representative images of the assay shown in FIG. 7 for wildtype (WT) and SIRPa KO #19 showing increase in puncta/areas of cancer cell phagocytosis.

FIG. 9 shows KO of SIRPa in engineered PSC-macrophages dramatically increases cancer cell phagocytosis with inclusion of EGFR opsonizing antibody. This suggests that SIRPa KO imparts functionality, and that general opsonization can now promote phagocytosis without CD47-opsonizing antibody.

FIG. 10 shows non-limiting, representative images of the assay shown in FIG. 9 for wildtype (WT) and SIRPa KO #19 showing increase in puncta/areas of cancer cell phagocytosis.

FIG. 11 shows a summary of all conditions from FIGS. 5, 7, and 9 for direct comparison.

FIG. 12 is an exemplary schematic showing how antibody opsonization promotes phagocytosis. In some embodiments using wild-type/primary macrophages, the antibody (or combination of antibodies) must include CD47 antibody to block the SIRPa signaling axis.

FIG. 13 is an exemplary schematic showing the utility of engineered macrophages. Without the need for CD47 antibody opsonization, preloading of KO macrophages with any IgG antibody can be used to generate combination therapeutics that make engineered macrophages cancer lineage specific.

FIG. 14 shows that CAR-P constructs can be targeted to the AAVS1 safe harbor locus in PSCs and that genetically modified PSCs can be differentiated into macrophages expressing the CAR-P constructs.

FIG. 15 shows an exemplary experimental setup to test functionality of PSC-CAR Macs.

FIG. 16 shows results of an exemplary validation test showing that the engineered DLD-1 cells described herein express CD19, the antigen for the CARs constructs described in FIG. 15.

FIG. 17 shows that CAR-P macrophages show some baseline activity in CD19-negative cells without the application of CD47 antibody.

FIG. 18 shows that CAR-P macrophages show some baseline activity in CD19-positive cells without the application of CD47 antibody.

FIG. 19 shows that CAR-P macrophages show some increased activity in CD19-negative cells with the application of CD47 antibody.

FIG. 20 shows that CAR-P macrophages show increased activity in CD19-positive cells with the application of CD47 antibody. These collective results show that the PSCs can be engineered with CARs that stimulate macrophage phagocytosis, and that macrophages derived from these engineered immune cells are functional towards the CAR antigen.

FIG. 21 is a schematic showing the development of a library of CARs with varying intracellular signaling pathways to stimulate phagocytosis without the need for opsonization.

FIG. 22 is a schematic showing an exemplary dual receptor sensing circuit using synthetic Notch.

FIG. 23 is a schematic showing an exemplary syn notch CAR targeting vector to test functionality in PSC-derived macrophages with antigen-responsive cell signaling to promote macrophage homing and migration to CAR antigen-specific cancer cells.

FIG. 24A-24B illustrating the different phagocytosis enhancement of stem cell-derived macrophages with respect to chimeric antigen receptor (CAR) design using a flow-cytometry-based competition assay. FIG. 24A illustrates cells treated without CD47 opsonizing antibodies and FIG. 24B illustrates cells treated with CD47 opsonizing antibodies.

### DETAILED DESCRIPTION

The present disclosure relates to engineered immune cells, compositions comprising said cells and methods thereof. In some aspects, the present disclosure relates to engineered macrophages derived from pluripotent stem cells. In some embodiments, the stem cell's genome is edited to knock-out (e.g., SIRPa) and/or knock-in (e.g., CAR) genes of interest prior to differentiation into macrophages. In some embodiments, the engineered macrophages lack a SIRPa receptor. In some embodiments, the engineered macrophages express chimeric antigen receptors that induce and/or enhance phagocytosis. Alternatively, or additionally, in some embodiments, macrophages are engineered with combinatorial antigen-sensing circuits to improve tumor recognition and phagocytosis. Combinations are also possible (e.g., cells engineered to delete SIRPa expression and induce CARP expression). Other aspects of the disclosure relate to methods of treating a disease (e.g., cancer) in a subject, and methods of enhancing phagocytosis of diseased cells by the engineered immune cells (e.g., macrophages).

In some aspects, the engineered cells as described herein enhance phagocytosis of cancer cells and/or are useful in the treatment of a disease (e.g., cancer). Without wishing to be bound by any particular theory, it is believed that editing the genome to knockout select genes (e.g., SIRPa gene) and knock-in select genes (e.g., synNotch, CAR-P, etc.) prior to cell differentiation (e.g., into macrophages), provides the engineered cells with enhanced functionality relative to native cells (e.g., non-engineered immune cells). For example, in some embodiments, the engineered cells (e.g., macrophages) described herein exhibit increased phagocytosis toward cancer cells, increased phagocytosis in response to IgG opsonization (as opposed to CD47 opsonization), increased phagocytosis in response to CAR-Ps, and/or enhanced tumor recognition abilities (e.g. combinatorial antigen-sensing circuits).

The disclosed engineered immune cells may be used as a standalone therapeutic, for example, for the treatment of cancer; alternatively, or additionally, the engineered immune cells (e.g., macrophages) may be used as an adjuvant (e.g., delivered after primary treatment such as surgery) and/or neoadjuvant (e.g., delivered before primary treatment such as surgery) therapy. In another set of embodiments, the macrophages of the current disclosure may be used concurrently with other treatment modalities (e.g., surgery, chemotherapy, radiation, immunotherapy, etc.).

Other applications are also contemplated. For example, in some embodiments, the engineered cells (e.g., macrophages) of the present invention may be used to phagocytize any target (e.g., cell, virus, bacteria, etc.) that leverages the CD47-SIRPa “do not eat me” signaling axis to gain immune privilege. Non-limiting examples include, but are not limited to, infection disease, inflammatory diseases (e.g., Crohn's disease, rheumatoid arthritis, irritable bowel syndrome, etc.), proliferative diseases, and autoimmune disease.

### Compositions Comprising Engineered Immune Cells

Aspects of the present disclosure relate to engineered immune cells (e.g., macrophages) or populations of engineered immune cells with increased phagocytosis and/or enhanced cell recognition capabilities (e.g., tumor cell recognition). In some embodiments, the immune cells are derived from pluripotent stem cells (e.g., human pluripotent stem cells, hPSC), a primary cell, or a non-stem cell derived immune cell. Pluripotent stem cells may be true embryonic stem cells (ES cells, e.g.,. are derived from human embryos or human fetal tissue), induced pluripotent stem cells (iPSC cells), somatic cell nuclear transfer cells (ntES cells), or parthenogenetic embryonic stem cells (pES cells). In some embodiments, the primary cell is a macrophage. In some cases, the genome of the stem cells is edited (e.g., knock-out and/or knock-in genes of interest) prior to differentiation into immune cells. In some cases, the primary cell genome is edited (e.g., to knock-out/down and/or knock-in genes of interest). The PSC cells may be differentiated into any suitable immune cell known in the art. Non-limiting examples include neutrophils, eosinophils, basophils, mast cells, monocytes, macrophages, natural killer cells (e.g., NK cells), dendritic cells, and lymphocytes (e.g., T cells and B cells). In a preferred set of embodiments, the PSC cells are differentiated into macrophages. In another set of embodiments The following is a non-limiting discussion of the various types of engineered immune cells contemplated herein.

### SIRPa-Knockout Macrophages

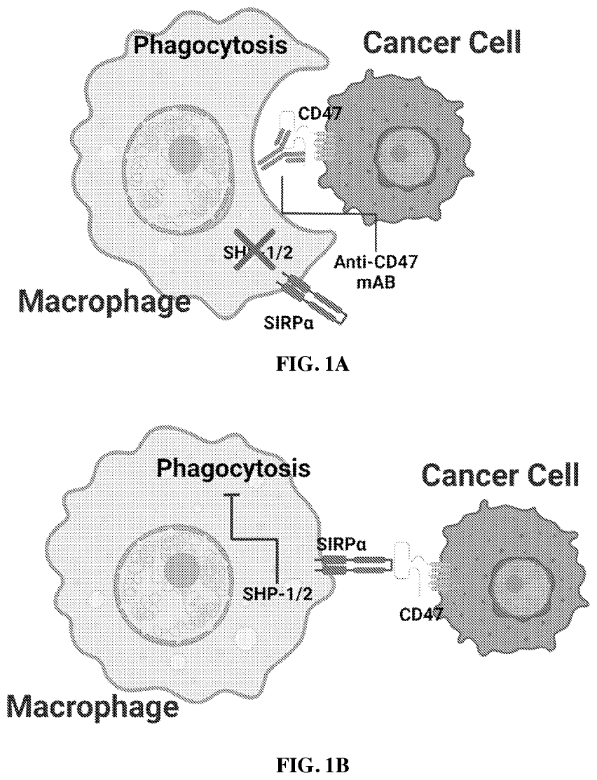

In some embodiments, PSC-Macs are engineered to reduce and/or eliminate expression of a SIRPa receptor (herein PSC-Mac-SIRPa-KO). In some embodiments, the PSC-Macs are engineered to eliminate surface expression of SIRPa. FIG. 1 shows a schematic representation illustrating the role of the CD47-SIRPa signaling axis in macrophage-mediated phagocytosis. Without wishing to be bound by any particular theory, it is believed that upon binding CD47 (e.g., on cancer cells), SIRPa initiates a signaling cascade that results in the inhibition of phagocytosis. This “don't eat me” signal is transmitted by phosphorylation of the immunoreceptor tyrosine-based inhibition motifs present on the cytoplasmic tail of SIRPa. Subsequent binding and activation of SHP-1 and SHP-2 blocks phagocytosis. This signaling cascade may be blocked by administration of CD47 antibodies that bind to CD47 receptors on cancer cells, which prevents binding and activation of the CD47-SIRPa signaling pathway and also provides a stimulus for macrophage phagocytosis via binding of the Fc domain on the CD47 antibody to the Fc receptor on the macrophage surface.

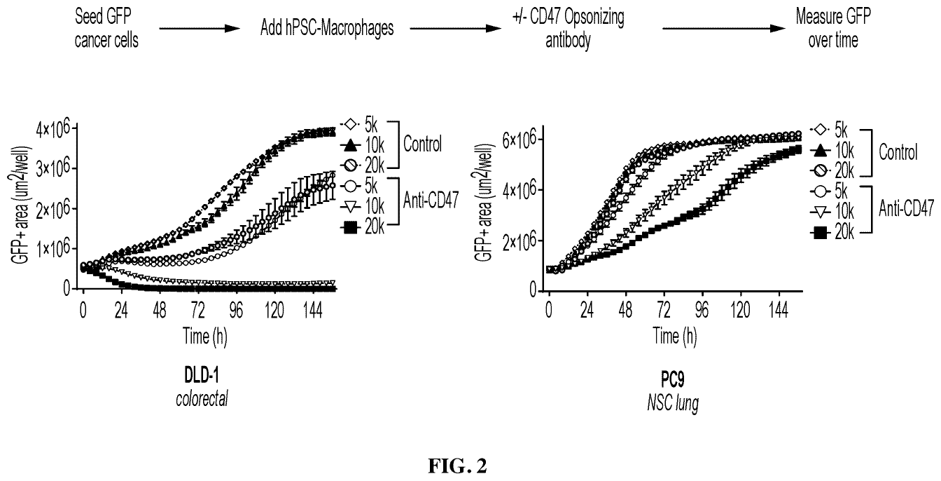

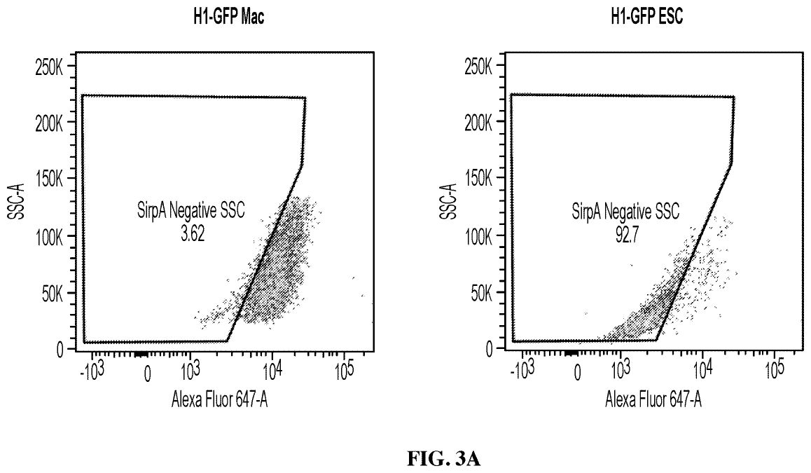

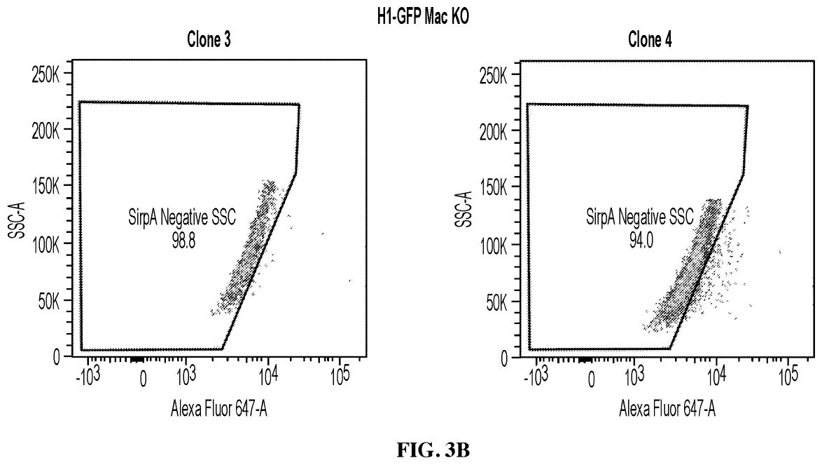

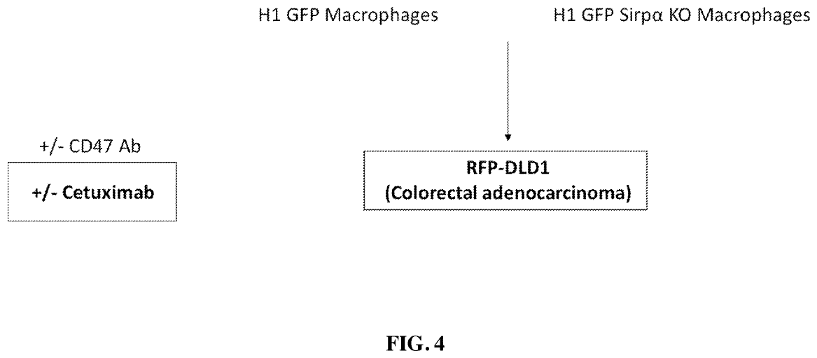

For example, FIG. 2 shows the limited ability of PSC-Macs expressing the SIRPa receptor to phagocytize whole solid tumor cells (e.g., DLD-1 colorectal and PC9 NSC lung cancer cells) in vitro. FIG. 2 also shows that the addition of a CD47 antibody to the in vitro culture significantly increases PSC-Mac phagocytosis, regardless of the starting macrophage culture density.

Without wishing to be bound by any particular theory, it is generally believed that editing the genome of the engineered immune cell (e.g., PSC-Macs) to eliminate expression of the SIRPa receptor (e.g., PSC-Macs-SIRPa-KOs) may circumvent the CD47-SIRPa signaling axis by preventing the engineered immune cell (e. g, macrophages) from receiving the “do not eat me” signal, thereby resulting in increased phagocytosis.

Thus, in some embodiments, the PSC genome is edited to render the SIRPa gene inoperative. The skilled artisan may use any technique known in the art to edit the genome in this manner. Non-limiting examples of methods used to knock out genes of interest include, but are not limited to, homologous recombination, site-specific nucleases (e.g., zinc-fingers, TALENS, and/or CRISPR/Cas9), and or mutagenesis (e.g., truncation mutants). In some embodiments, the genome may be edited in a coding region and/or a non-coding region of the genome. In some embodiments, editing the genome results in 100% knockout of the target gene. In other embodiments, editing the genome results in less than 100% knockout of the target gene (e.g., between 50% and 90%). A more detail description of methods used to produce PSC-Mac-SIRPa-KOs are described elsewhere herein.

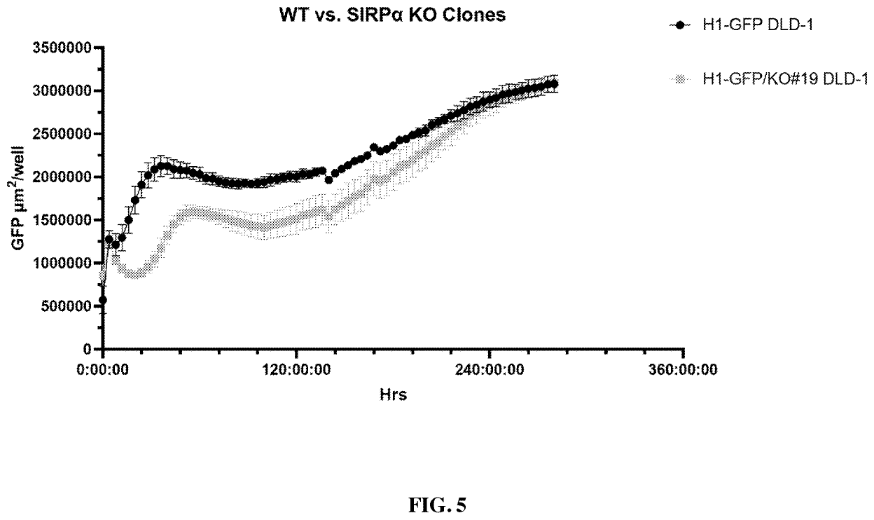

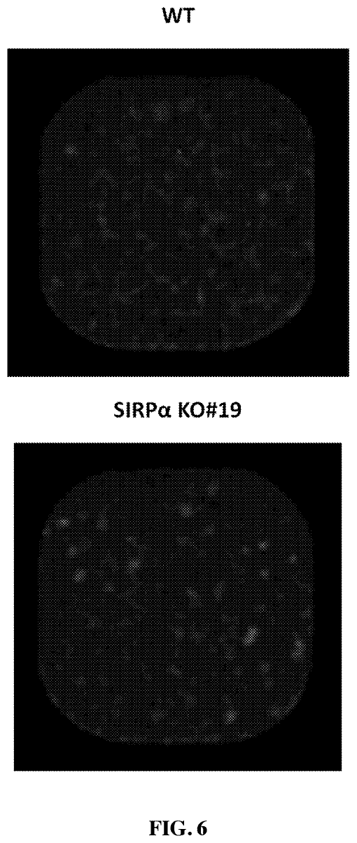

FIGS. 5 and 6 illustrate the phagocytosis of PSC-Macs and PSC-Mac-SIRPa-KOs against DLD-1 adenocarcinoma cancer cells. As illustrated in FIG. 5, the phagocytosis of the KOs is about 44% greater than PSC-Macs after about 36 hours of culture. Interestingly, the phagocytosis of the KOs decreased with increasing culture time (e.g., PSC-Macs and PSC-Mac-SPIRa-KOs exhibit similar phagocytosis after 240 hours of culture).

Thus, in some embodiments, knocking out the SIRPa gene increases the phagocytosis of the engineered cell (e.g., macrophages) compared to non-edited cell (e.g., macrophages). In some embodiments, knocking down the SIRPa gene increases the phagocytosis by greater than or equal to 1%, greater than or equal to 5%, greater than or equal to 10%, greater than or equal to 15%, greater than or equal to 20%, greater than or equal to 25%, greater than or equal to 30%, greater than or equal to 35%, greater than or equal to 40%, and greater than or equal to 45%, relative to non-engineered cells. In some embodiments, knocking down the SIRPa gene increases the phagocytosis by less than or equal to 45%, less than or equal to 40%, less than or equal to 35%, less than or equal to 30%, less than or equal to 25%, less than or equal to 20%, less than or equal to 15%, less than or equal to 10%, less than or equal to 5%, and less than or equal to 1%, relative to non-engineered cells.

Some aspects of the disclosure relate to a targeted agent (e.g., an antibody). In some embodiments, the targeted agent comprises an antibody, antibody derivative, and/or fragment thereof In some embodiments, the antibody targets the CD47-SIRPa signaling axis via directly binding to either SIRPa receptors on engineered immune cell surface or CD47 ligands on the target cell surface (e.g., cancer cell surface).

Without wishing to be bound by any particular theory, it is generally believed that the CD47 antibody inhibits the CD47-SIRPa signaling pathway and/or stimulates opsonization (i.e., it is an opsonin). Opsonins are substances (e.g., antibodies) which bind to epitopes (e.g., antigens) on cell surfaces and serve as an “eat me” signal to phagocytes (i.e., it marks them for phagocytosis via opsonization). Antibodies (e.g., anti-CD47, IgG, IgA, IgM, IgE, etc.) comprise a Fab domain which allows them to bind to antigens on the surface of cancer cells, and an Fc domain which allows them to bind to, and activate, phagocytotic cells (e.g., macrophages) via their Fc receptor, thus stimulating the phagocytotic cell (e.g., macrophages) to eat the cancer cell. Other compounds may also serve as opsonins such as, for example, the complement proteins C3b and C4b.

In some embodiments, the engineered immune cell comprises an Fc receptor configured to bind to a targeted agent (e.g., antibody, an antibody derivative, and/or any fragment thereof) comprising an Fc domain. In some embodiments, the Fc receptor (e.g., FcγRi, ReγRIIA, FcγRIIB1, FcγRIIB2, FcγRIIIA, FcγRIIIB, FcRn) of the engineered immune cells binds to an Fc domain on an IgG isotype antibody. In some embodiments, the Fc receptor (e.g., FcεRI, FcεRII) of the engineered immune cells binds to an Fe domain on an IgE isotype antibody. In some embodiments, the Fc receptor (e.g., FcαRI and Fcα/μR) of the engineered immune cells binds to an Fc domain on an IgA isotype antibody. In some embodiments, the Fc receptor (e.g., Fcα/μR) of the engineered immune cells binds to an Fc domain on an IgM isotype antibody.

In some embodiments, the targeted agent is an antibody, an antibody derivative, and/or any fragment thereof configured to stimulate phagocytosis (e.g., antibody-mediated phagocytosis). Without wishing to be bound by theory, it is believed that the antibody, antibody derivative, and/or any fragment thereof may stimulate phagocytosis either directly or indirectly. For example, in some cases, the antibody, antibody derivative, and/or any fragment thereof comprises a Fab domain configured to bind to a surface receptor on a cancer cell (e.g., a tumor associated antigens) and an Fc domain configured to bind to an Fc receptor on an immune cell. This allows the immune cell to directly phagocytose the labeled cancer cell. Alternatively, or additionally, the antibody, antibody derivative, and/or fragment thereof, comprises a Fab domain that is not configured to bind to a surface receptor on a cancer cell, but does comprise an Fc domain configured to bind to the Fc domain of an immune cell. In this scenario, the immune cell may be primed to phagocytize a cell, but requires an alternative way to bind to a target tumor cells, such as, for example, the CAR constructs and/or synNotch constructs described elsewhere herein. Non-limiting examples of antibody, antibody derivatives, and/or fragments thereof include the IgG derived monoclonal antibodies rituximab, panitumumab, trastuzumab, daratumumab, and dinutuximab. Other antibodies may also be used.

In some embodiments, the targeted agent is an antibody, an antibody derivative, and/or any fragment thereof configured to stimulate phagocytosis (e.g., antibody-mediated phagocytosis). Without wishing to be bound by theory, it is believed that the antibody, antibody derivative, and/or any fragment thereof may stimulate phagocytosis either directly or indirectly. For example, in some cases, the antibody, antibody derivative, and/or any fragment thereof comprises a domain, e.g., a Fab domain, configured to bind to a surface receptor on a cancer cell (e.g., a tumor associated antigens), and an Fc domain configured to bind to an Fe receptor on an immune cell. This allows the immune cell to directly phagocytose the labeled cancer cell. Alternatively, or additionally, the antibody, antibody derivative, and/or fragment thereof, comprises a Fab domain that is not configured to bind to a surface receptor on a cancer cell, or does not comprise a Fab domain, but does comprise an Fc domain configured to bind to the Fc domain of an immune cell. In this scenario, the immune cell may be primed to phagocytize a cell, but in some embodiments requires an alternative way to bind to a target tumor cells, such as, for example, the CAR constructs and/or synNotch constructs described elsewhere herein. Non-limiting examples of antibody, antibody derivatives, and/or fragments thereof include the IgG derived monoclonal antibodies rituximab, panitumumab, trastuzumab, daratumumab, and dinutuximab. Other antibodies may also be used.

In some embodiments, the targeted agent comprises an IgG, IgA, IgE, and/or IgM antibody, and/or any subclasses thereof, and/or any derivatives thereof, and/or and fragments

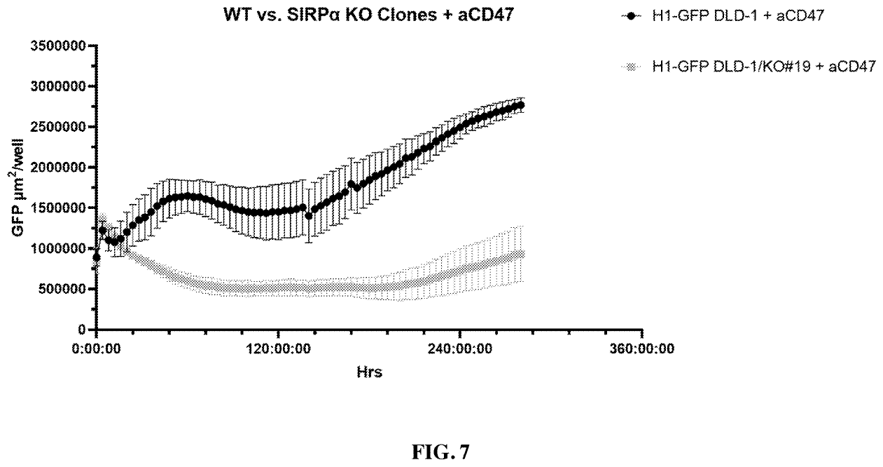

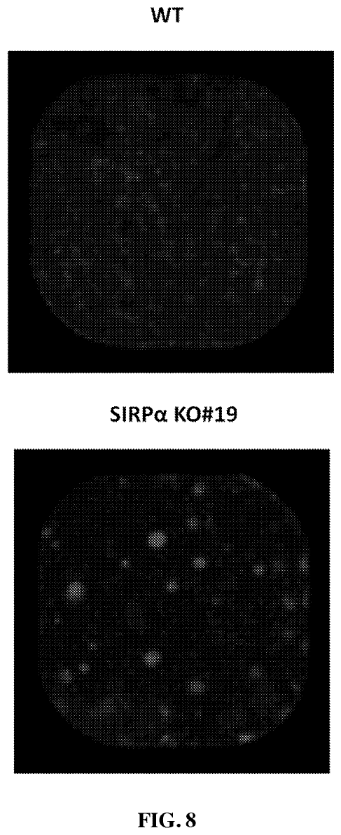

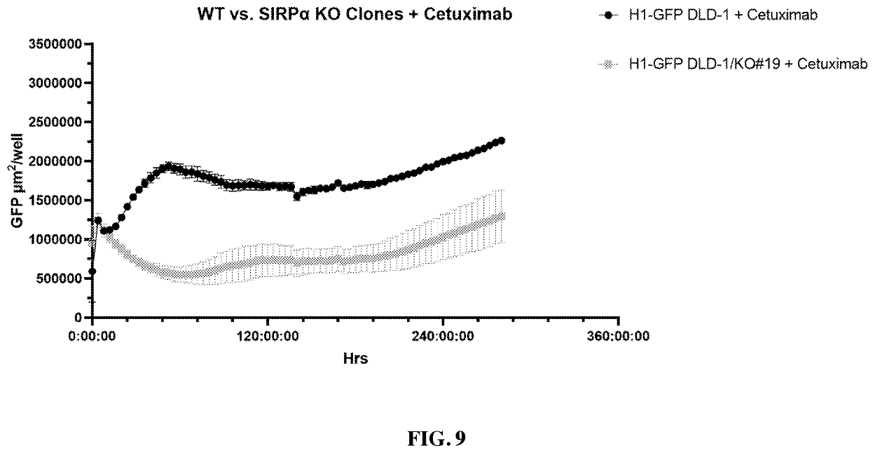

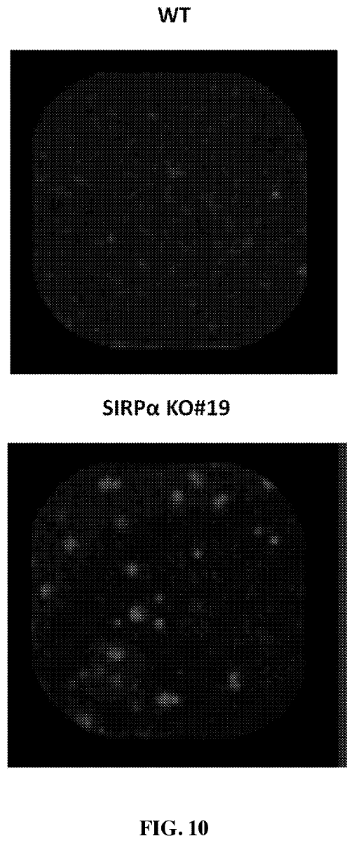

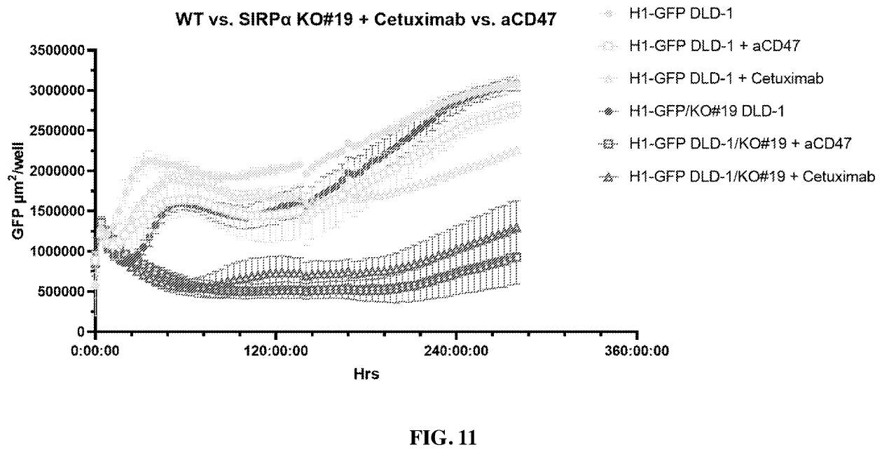

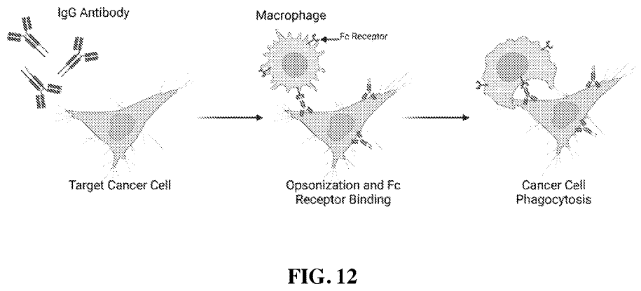

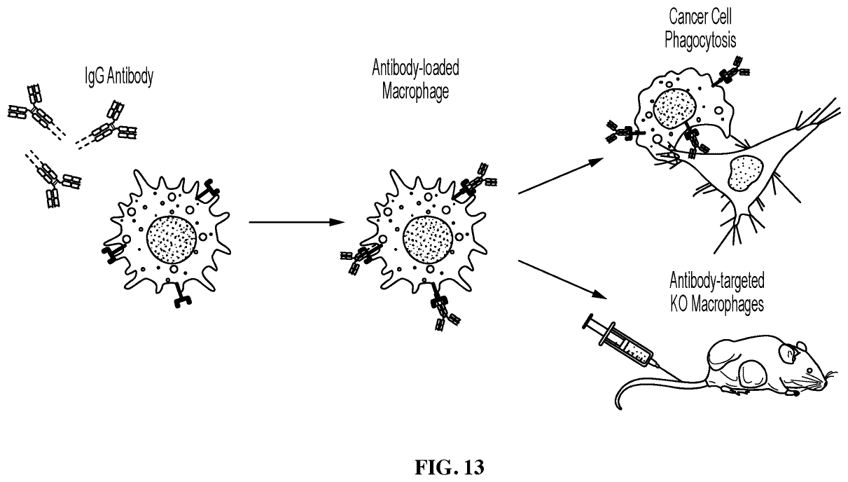

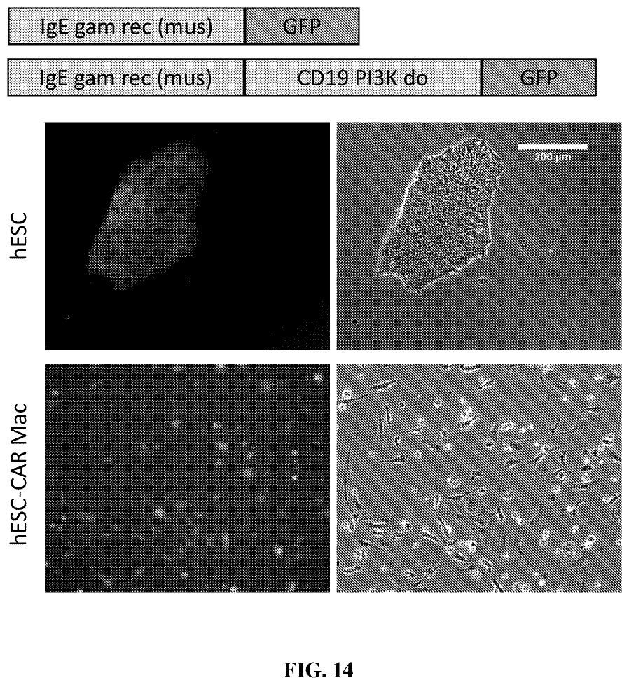

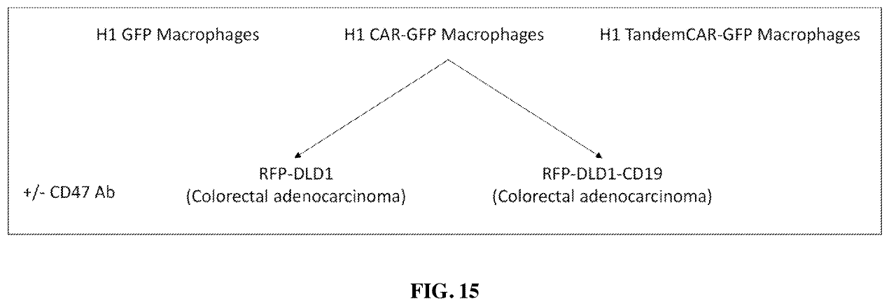

In some embodiments, the targeted agent is a CD47 antibody. CD47 antibodies are art recognized therapeutics capable of disrupting the CD47-SIRPa signaling axis. For example, FIGS. 7 and 8 illustrate the number of cells phagocytized following treatment with compositions comprising PSC-Macs and CD47 antibody and PSC-Mac-SIRPa-KOs and CD47 antibody against DLD-1 adenocarcinoma cancer cells. As illustrated in FIG. 7, the phagocytosis observed using the PSC-Mac-SIRPa-KOs and CD47 antibody is about 60% greater than the PSC-Mac and CD 47 antibody after between 120 and 360 hours of culture. In some embodiments, the CD47 antibody is an IgG isotype.

In some embodiments, the targeted agent is any agent known to one of skill in the art capable of interrupting the CD47-SIRPa signaling axis. Non-limiting exemplary compounds are discussed in detail in Maute et al., “CD47-SIRPa-targeted therapeutics: status and prospects” Immuno-Oncology and Technology; 13, March 2022, 100070. For example, in some cases, the targeted agent may be a monoclonal antibody or a polyclonal antibody. In some embodiments, the targeted agent is a monoclonal antibody to CD47 (e.g., magrolimab, lemzoparlimab, SRF231, CC-90002, AO-176,, a monoclonal antibody to SIRPa, or a monoclonal antibody to a SIRPa fusion protein. In some cases, the targeted agent a small molecule, an antibody, a peptide, a protein, a polypeptide, a polynucleotide, an aptamer, a PNA, or the like. In some embodiments the targeted agent is a combination of one or more agents described herein.

In some embodiments, combinations of engineered immune cells and/or targeted agents are possible. For example, in some cases, the combination of engineered immune cells (e.g., PSC-Mac-SIRPa-KO cells) and targeted agents (e.g., CD47 antibody) increases the engineered immune cell's ability to recognize and kill the cells targeted by the targeted agent relative to non-engineered immune cells alone (e.g., lacking targeted agents). In some embodiments, a composition comprising the engineered immune cells (e.g.,. PSC-Mac-SIRPa-KO) and/or a targeted agent (e.g., CD47 antibody) may increase the phagocytosis by greater than or equal to 1%, greater than or equal to 5%, greater than or equal to 10%, greater than or equal to 15%, greater than or equal to 20%, greater than or equal to 25%, greater than or equal to 30%, greater than or equal to 35%, greater than or equal to 40%, greater than or equal to 45%, greater than or equal to 50%, greater than or equal to 55%, and greater than or equal to 60%, relative to compositions comprising non-engineered immune cells (e.g., PSC-Macs) and a targeted agent (e.g., CD47 antibody). In some embodiments, compositions comprising an engineered immune cells (e.g., PSC-Mac-SIRPa-KO) and/or a targeted agent (e.g., CD47 antibody) increase the phagocytosis by less than or equal to 60%, less than or equal to 55%, less than or equal to 50%, less than or equal to 45%, less than or equal to 40%, less than or equal to 35%, less than or equal to 30%, less than or equal to 25%, less than or equal to 20%, less than or equal to 15%, less than or equal to 10%, less than or equal to 5%, and less than or equal to 1%, relative to compositions comprising non-engineered immune cells (e.g., PSC-Macs) and a targeted agent (e.g., CD47 antibody). Other combinations are also possible.

In some embodiments, the targeted agent is an antibody against a tumor associated antigen (TAA), such as those described by Leko et al in “Identifying and targeting human tumor antigens for T cell-based immunotherapy of solid tumors” Cancer Cell, 38, Oct. 12, 2020. Without wishing to be bound by any particular theory, TAAs are known antigenic substances produced by tumor cells and may serve as a target for opsonization. Examples of TAAs include, but are not limited to, alphafetoprotein (AFP), carcinoembryonic antigen (CEN), CA-125, MUC-1, epithelial tumor antigen (ETA), tyrosinase, and melanoma-associated antigen (MAGE), among others.

In some embodiments, the targeted agent (e.g., CD47 antibody and/or other IgG antibody) may be administered to a subject in need thereof separately from the engineered immune cell. For example, in some cases, the targeted (e.g., IgG antibody) may be delivered before and/or after administration of the engineered immune cell (e.g., PSC-Mac-SIRPa-KO cells). In some embodiments, the targeted agent (e.g., CD47 antibody and/or IgG antibody) is delivered before administration of the hPSC-Mac-SIRPa-KO cells. In some embodiments, the targeted agent (e.g., CD47 antibody and/or IgG antibody) is delivered at the same time. Alternatively, or additionally, the targeted agent (e.g., IgG antibody) may be produced by the subject receiving the pharmaceutical composition. For example, it is generally accepted in the art that IgG antibodies represent approximately 75% of all serum antibodies in humans and are the most common type of antibody found in blood circulation. Thus, in some embodiments, a subjects own IgG antibodies may opsonize the tumor cells, thus marking them for phagocytosis.

In some embodiments, genes other than, or in addition to, SIRPa, may be knocked out. Any gene of interest capable of being knocked down may be knocked down using methods known in the art. In some embodiments, gene targets of interest include macrophage inhibitory receptors. Non-limiting exemplary embodiments include CD47, SIRPα, EGFR, MHC I, CD24, CALR, CD40, PD-L1, APMAP, GPR84, VCAM1, CD11b, SIGLEC-10, PD-L2, PD-1, CD73, epCAM, Galectin-9, CD14, CD80, CD86, SIRPb, SIRPg, SLAMF7, MARCO, AXL, CLEVER-1, ILT4, TIM-3, TIM-4, LRP-1, calreticulin, TREM1, TREM2, GD2, FcgRI, FcgRIIa, FcgRIIb, FcgRIII, MUCI, CD44, CD63, CD36, CD84, CD164, CD82, CD18, SIGLEC-7, CD166, CD39, CD46, LILRA1, LILRA2(ILT1), LILRA3 (ILT6), LILRA4 (ILT7), LILRB1 (ILT2), LILRB2 (ILT4), LILRB3 (ILT5), LILRB4 (ILT3), LILRB5, CD85b (ILT8 or ILT9), CD85m (ILT10), CD85f (ILT11), CD276, CD88, CD99, PILRa, Siglec-9, CD206, CD163, CD84 (SLAMF5), C3aR, or CLEC12A. Other genes and/or combinations of genes may also be knocked out in some embodiments.

In some embodiments, the immune cell genome is edited to knockdown or knockout expression of one or more target genes. In some cases, the one or more target genes may be expressed on the surface of the cell. Thus, in some embodiments, the immune cell genome is edited to knockdown or knockout a surface receptor or surface ligand. In some cases, the knocked down or knocked out surface receptor is SIRPa

In some embodiments, the immune cell genome (e.g., primary macrophage or stem cell derived macrophage) is edited to knock-down a gene of interest (e.g., SIRPa) without completely eliminating expression of said gene (e.g. surface expression of SIRPa). For example, in some embodiments, a primary macrophage (e.g., obtained via differentiation of primary blood monocytes) is edited (e.g., using RNAi) to transiently knock-down a target gene (e.g., SIRPa). Alternatively, or additionally, in some embodiments, the immune cell may be edited to knock-out a target gene (e.g., SIRPa). In some embodiments, the gene product may be expressed on the surface of the immune cell. In some cases, the immune cell may be edited to knock-down expression of said target gene (e.g., SIRPa) without completely eliminating surface expression of said gene product.

In some embodiments, a gene may be knocked-out in an immune cell and the gene product may be expressed at greater than or equal to 1%, greater than or equal to 5%, greater than or equal to 10%, greater than or equal to 20%, greater than or equal to 30%, greater than or equal to 40%, or greater than or equal to 50% of the non-edited immune cell. In some embodiments, the gene may be knocked-out in an immune cell and the gene product may be expressed at less than or equal to 50%, less than or equal to 40%, less than or equal to 30%, less than or equal to 20%, less than or equal to 10%, less than or equal to 5%, or less than or equal to 1% of the non-edited immune cell.

In some embodiments, the genome of the immune cell (e.g., primary macrophage or PSC) is edited to disrupt the function of a target gene (e.g., SIRPa gene). For example, in some cases, the target gene is a surface receptor (e.g., SIRPa) configured to send inhibitory signals to the cell (e.g., anti-inflammatory signals) following binding to its conjugate ligand. In some cases, the gene function may be disrupted by truncating or mutating one or more of the gene domains (e.g., an intracellular domain, extracellular, transmembrane, etc.). For instance, truncation of the intracellular domain of the SIRPa gene, permits binding of the extracellular domain to CD47, but inhibits transmission of the intracellular inhibitory signals. Alternatively, or additionally, mutagenesis of the extracellular domain, for example, may reduce the binding affinity of the SIRPa receptor for CD47, also leading to decreased inhibitory signaling. Other embodiments are also possible. For example, in some cases it may be possible to mutate an intracellular domain (e.g., ITIM motifs on intracellular domain of SIRPa) of a gene target that inhibits intracellular signaling, but does not affect surface expression of the target receptor. Combinations are also possible. For instance, in some instances, an intracellular and an extracellular domain may be edited to further enhance disruption of the target gene.

### Chimeric Antigen Receptor (CAR) Macrophages

In some embodiments, the disclosure relates to an engineered immune cell comprising a genome that has been edited to knock-in one or more genes of interest (e.g., chimeric antigen receptor genes) prior to differentiation (e.g., into macrophages). For example, in one set of embodiments, the engineered immune cell is derived from a PSC whose genome is edited to enable expression of one or more chimeric antigen receptors configured to stimulate phagocytosis (herein PSC-Mac-CAR). Without wishing to be bound by any particular theory, it is believed engineered immune cells expressing CAR-P constructs may exhibit increased targeting and killing of cells bearing the chimeric antigen of interest without the need for an “eat me” signal (e.g., opsonization). Like their CAR-T cell counterparts, CAR-Ps comprise a target binding extracellular domain (e.g., scFv domain), followed by a hinge sequence, a transmembrane domain, and an intracellular signaling domain. The extracellular domain permits tumor cell recognition (via binding to TAAs) while the intracellular signaling domain stimulates phagocytosis. In some embodiments, the extracellular domain comprises a signaling peptide and an antigen recognition region. The signal peptide directs nascent protein into the endoplasmic reticulum. In some embodiments, the signal peptide is derived from CD28. The antigen recognition region (also known as the single-chain variable fragment, scFv) comprises a fusion protein or a chimeric protein. In some embodiments, the scFv comprises a chimeric protein made up of light and heavy chains of immunoglobins connected with a short linker peptide. Exemplary targets (e.g., antigens) include, but are not limited to, BCMA, CD19, CD22, CD20, CD138, CD33, CD123, PSMA, Igk, LeY, ROR1, biotin, CD171, EGFRVIII, FAP, FR, glypican-3, HER2, MUC-1, IL13Ra2, mesothelin, NKG2D, PD1, CD47, SIRPα, EGFR, MHC I, CD24, CALR, CD40, PD-L1, APMAP, GPR84, VCAM1, CD11b, SIGLEC-10, PD-L2, PD-1, CD73, epCAM, Galectin-9, CD14, CD80, CD86, SIRPb, SIRPg, SLAMF7, MARCO, AXL, CLEVER-1, ILT4, TIM-3, TIM-4, LRP-1, calreticulin, TREM1, TREM2, GD2, FcgRI, FcgRIIa, FcgRIIb, FcgRIII, MUC1, CD44, CD63, CD36, CD84, CD164, CD82, CD18, SIGLEC-7, CD166, CD39, CD46, LILRA1, LILRA2 (ILT1), LILRA3 (ILT6), LILRA4 (ILT7), LILRBI (ILT2), LILRB2(ILT4), LILRB3 (ILT5), LILRB4(ILT3), LILRB5, CD85b (ILT8 or ILT9), CD85m (ILT10), CD85f (ILT11), CD276, CD88, CD99, PILRa, Siglec-9, CD206, CD163, CD84 (SLAMF5), C3aR, or CLEC12A

In some embodiments, the hinge sequence comprises hydrophilic residues with sections of glycine and serine for flexibility and sections of glutamate and lysine for added solubility. In some embodiments, the hinge domain is derived from CD3φ, CD4, CD8α, CD28, and combinations thereof. Other domains are also possible.

In some embodiments, the transmembrane comprises a hydrophobic alpha helix that spans the cell membrane and is essential for the stability of the receptor as a whole. In some embodiments, the transmembrane domain is derived from CD35, CD4, CD8α, CD28, and combinations thereof. Other domains are also possible.

In some embodiments, the intracellular signaling domain stimulates the PSC-Mac-CAR to phagocytize the target cell. Non-limiting examples of intracellular domains that trigger phagocytosis include Megf10, FcRγ, CD147, CD3φ, IgEφ rec (human), CD19 P13K do, CD28φ, 4-1BB, FCGR1, FCGR2, and any combination thereof. In some embodiments, the intracellular signaling domain comprises two or more domains linked in tandem, for example, to increase the frequency of whole cell engulfment. In some embodiments, the tandem constructs comprise a FcRy domain fused CD19 cytoplasmic domain. For example, in some embodiments, the tandem construct comprises amino acids 500-534 Mouse CD19 (Uniprot CD19_MOUSE) fused to amino acids 19-86 of Mouse Fc ERG precursor (FCERG MOUSE).

In some embodiments, the CAR constructs are integrated into the PSC genome at a safe harbor site (SHS). SHS are genomic locations where new genes or genetic elements can be introduced without disrupting the expression or regulation of adjacent genes. In some embodiments, the SHS include the canonical human SAS, AAVS1, CCR5, and hROSA26. In a preferred set of embodiments, the CAR genes are inserted into the AAVSI safer harbor locus.

Those of skill in the art will understand that the compositions described herein represent exemplary embodiments, and that any extracellular domain, hinge sequence, transmembrane domain, and/or intracellular signaling domain of interest, or combination thereof, may be incorporated into any SHS in the PSC genome of the macrophages described herein.

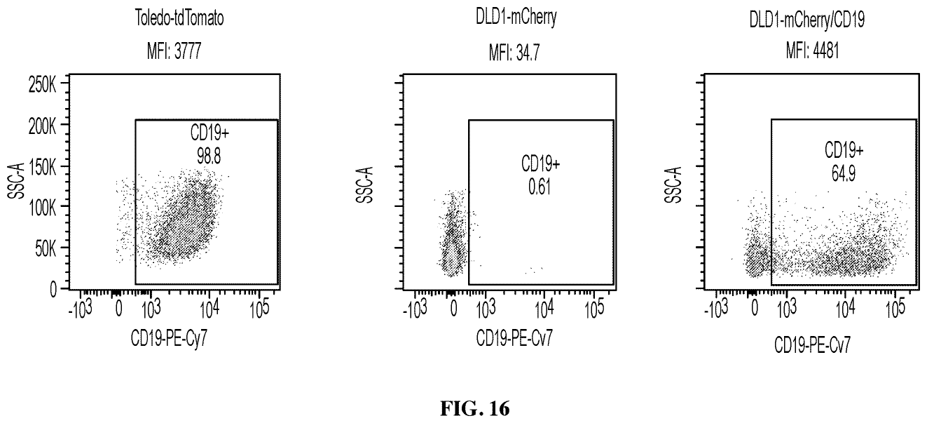

FIG. 16 illustrates the phagocytosis of PSC-Macs, PSC-Mac-CAR, and PSC-Mac-CAR (tandem) designed to target an antigen (e.g., CD19) against CD19 negative DLD-1 adenocarcinoma cancer cells. In some embodiments, the phagocytosis of the engineered immune cell (e.g., PSC-Mac-CARs) is about 30% greater than non-engineered immune cells (e.g., PSC-Mac) after about 240 hours of culture.

Thus, in some embodiments, engineered immune cells (e.g., PSC-Mac-CARs) described herein exhibit increased non-targeted phagocytosis (e.g., they phagocytize tumors lacking the target antigen) relative to non-engineered immune cells (e.g., PSC-Macs, macrophages lacking the CAR constructs). In some embodiments, the increased phagocytosis of engineered immune cells (e.g., PSC-Mac-CARs) is greater than or equal to 1%, greater than or equal to 5%, greater than or equal to 10%, greater than or equal to 15%, greater than or equal to 20%, greater than or equal to 25%, and greater than or equal to 30%, relative to non-engineered immune cells (e.g., PSC-Macs). In some embodiments, the increased phagocytosis of engineered immune cells (e.g., PSC-Mac-CARs) is less than or equal to 30%, less than or equal to 25%, less than or equal to 20%, less than or equal to 15%, less than or equal to 10%, less than or equal to 5%, and less than or equal to 1%, relative non-engineered immune cells (e.g., PSC-Macs).

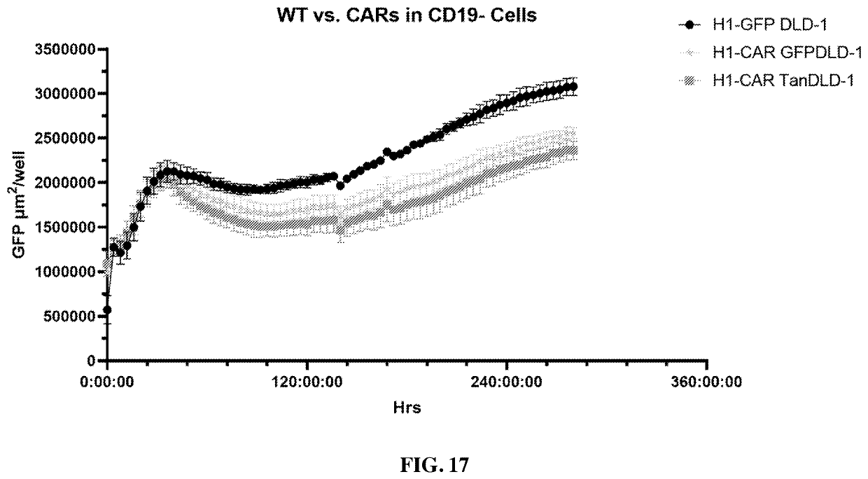

FIG. 17 illustrates nonlimiting examples of the ability of non-engineered immune cells (e.g., PSC-Macs) and engineered immune cells phagocytize (e. g, PSC-Mac-CAR and PSC-Mac-CAR (tandem)) to target CD19 against CD19 positive DLD-1 adenocarcinoma cancer cells. As illustrated in FIG. 17, the phagocytosis of the engineered immune cells (e.g., PSC-Mac-CARs is about 25% greater than the composition comprising the non-engineered immune cells (e.g., PSC-Mac) after about 240 hours of culture.

Thus, in some embodiments, the engineered immune cells (e.g., PSC-Mac-CARs) described herein exhibit increased targeted phagocytosis relative to non-engineered immune cells (e.g., PSC-Macs, macrophages lacking the CAR constructs). In some embodiments, the increased phagocytosis of engineered immune cells (e.g., PSC-Mac-CARs) is greater than or equal to 1%, greater than or equal to 5%, greater than or equal to 10%, greater than or equal to 15%, greater than or equal to 20%, and greater than or equal to 25%, relative to non-engineered immune cells (e.g., PSC-Macs). In some embodiments, the increased phagocytosis of engineered immune cells (e.g., PSC-Mac-CARs) is less than or equal to 25%, less than or equal to 20%, less than or equal to 15%, less than or equal to 10%, less than or equal to 5%, and less than or equal to 1%, relative non-engineered immune cells (e.g., PSC-Macs).

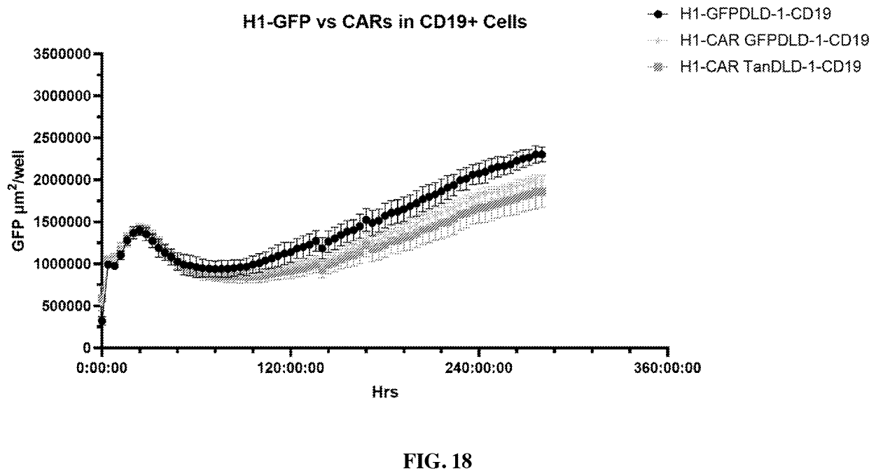

FIG. 18 illustrates the phagocytotic activity of a composition comprising a combination of engineered immune cells (e.g., PSC-Mac-CARs) and a targeted agent. More specifically, FIG. 18 shows compositions comprising PSC-Macs and CD47 antibody, PSC-Mac-CAR and CD47 antibody, and PSC-Mac-CAR(tandem) and CD47 antibody designed to target CD19 against CD19 negative DLD-1 adenocarcinoma cancer cells. As illustrated in FIG. 18, the phagocytosis exhibited by compositions comprising PSC-Mac-CAR and CD47 antibodies and PSC-Mac-CAR(tandem) and CD47 antibodies was about 40% and 60% greater than the compositions comprising the PSC-Mac cells after about 240 hours of culture.

Thus, in some embodiments, compositions comprising a combination of an engineered immune cells (e.g., PSC-Mac-CARs) and targeted agent (e.g., CD47 antibodies) exhibit increased non-targeted phagocytosis relative to non-engineered immune cells (e.g., PSC-Macs, macrophages lacking the CAR constructs). In some embodiments, the increased phagocytosis of compositions comprising engineering immune cells (e.g., PSC-Mac-CARs) and a targeted agent (e.g., CD47 antibody) is greater than or equal to 1%, greater than or equal to 5%, greater than or equal to 10%, greater than or equal to 15%, greater than or equal to 20%, greater than or equal to 25%, greater than or equal to 30%, greater than or equal to 35%, greater than or equal to 40%, greater than or equal to 45%, greater than or equal to 50%, greater than or equal to 55%, and greater than or equal to 60%, relative to compositions comprising non-engineered immune cells and a targeted agent (e.g., PSC-Macs/CD47). In some embodiments, the increased phagocytosis of compositions comprising engineered immune cells and a targeted agent (e.g., PSC-Mac-CARs/CD 47) is less than or equal to 60%, less than or equal to 55%, less than or equal to 50%, less than or equal to 45%, less than or equal to 40%, less than or equal to 35%, less than or equal to 30%, is less than or equal to 25%, less than or equal to 20%, less than or equal to 15%, less than or equal to 10%, less than or equal to 5%, and less than or equal to 1%, relative to compositions comprising non-engineered immune cells and targeted agents (e.g., PSC-Mac/CD47).

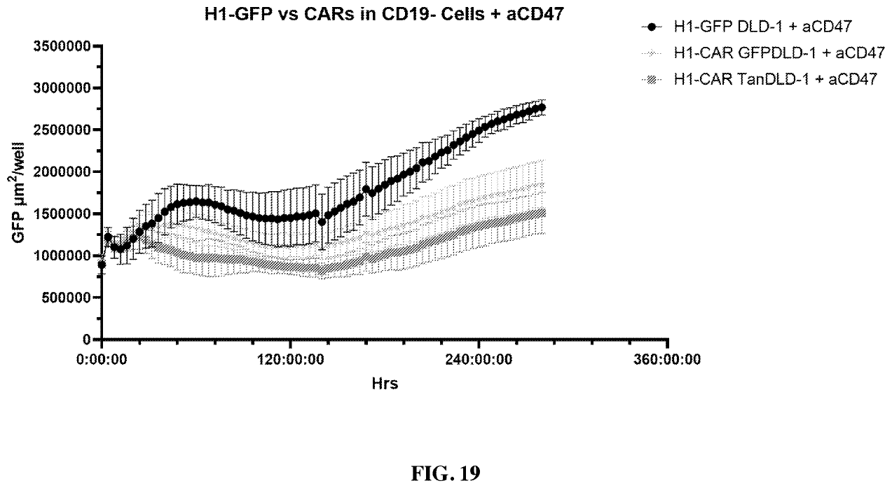

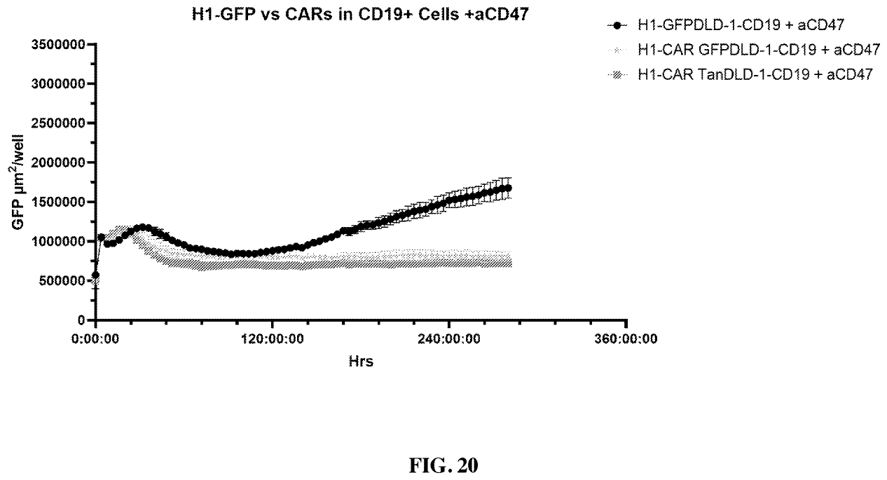

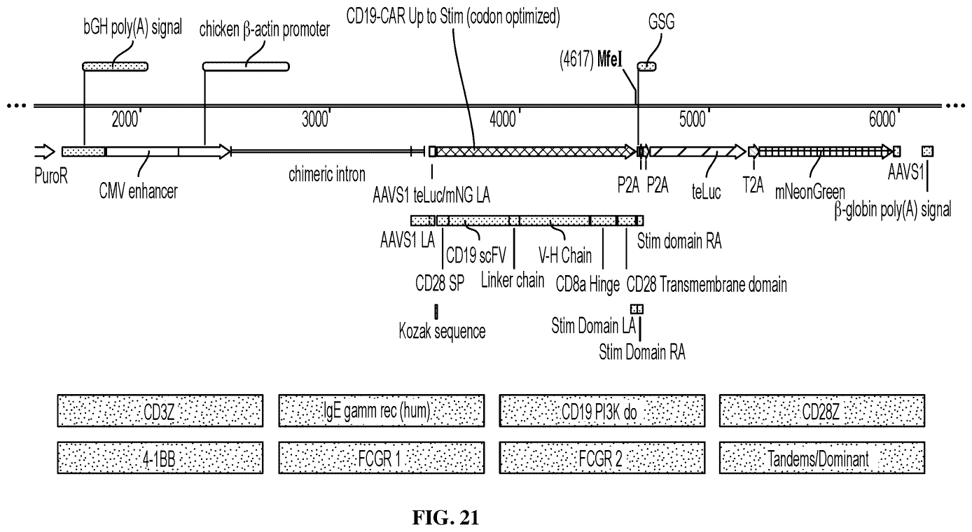

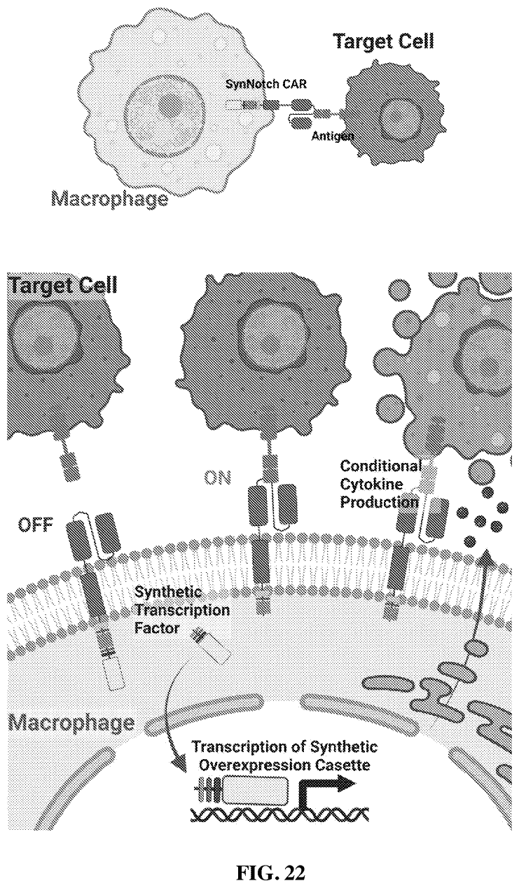

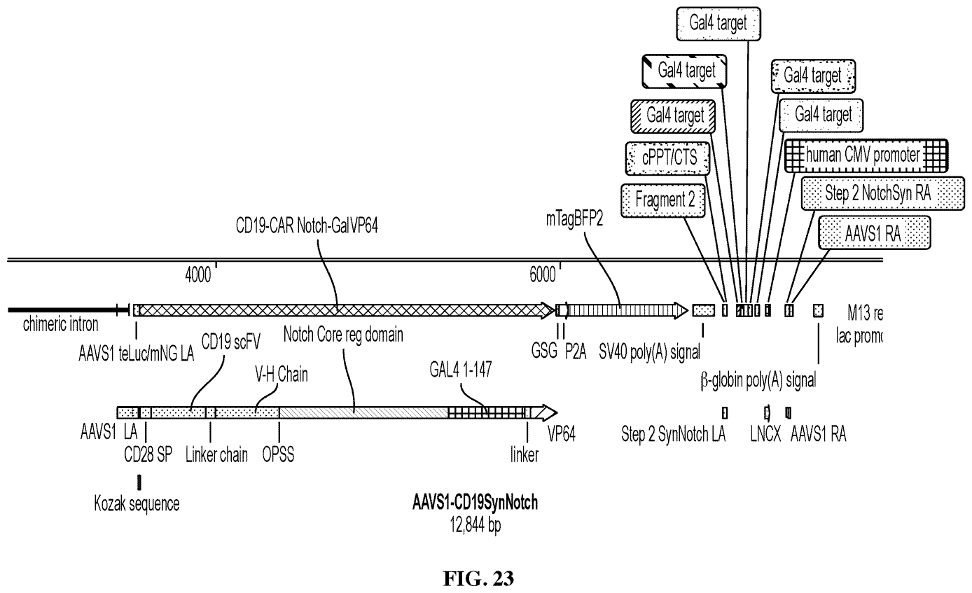

FIG. 19 illustrates the phagocytotic activity of compositions comprising a combination of engineered immune cells (e.g., PSC-Mac-CARs) and a targeted agent to compositions comprising non-engineered immune cells and the targeted agent. More specifically, FIG. 19 shows compositions comprising PSC-Macs and CD47 antibody, PSC-Mac-CAR and CD47 antibody and PSC-Mac-CAR(tandem) and CD47 antibody designed to target CD19 against CD19 positive DLD-1 adenocarcinoma cancer cells. As illustrated in FIG. 19, the phagocytosis exhibited by compositions comprising the engineered immune cells and targeted agents (e.g., PSC-Mac-CAR/CD47 antibody and PSC-Mac-CAR(tandem)/CD47 antibody) were both about 80% greater than the composition comprising the non-engineered immune cells and targeted agents (e.g., PSC-Mac/CD 47) after about 240 hours of culture.

Thus, in some embodiments, engineered immune cells (e.g., PSC-Mac-CARs) and targeted agents (e.g., CD47 antibodies) exhibit increased targeted phagocytosis relative to non-engineered immune cells (e.g., PSC-Macs, macrophages lacking the CAR constructs). In some embodiments, the increased phagocytosis of compositions comprising the engineered immune cells and targeted agent (e.g., PSC-Mac-CARs/CD 47) is greater than or equal to 1%, greater than or equal to 5%, greater than or equal to 10%, greater than or equal to 15%, greater than or equal to 20%, greater than or equal to 25%, greater than or equal to 30%, greater than or equal to 35%, greater than or equal to 40%, greater than or equal to 45%, greater than or equal to 50%, greater than or equal to 55%, greater than or equal to 60%, greater than or equal to 65%, greater than or equal to 70%, greater than or equal to 75%, and greater than or equal to 80% relative to compositions comprising non-engineered immune cells and targeted agent (e.g., PSC-Macs/CD47 antibody). In some embodiments, the increased phagocytosis of compositions comprising engineered immune cells and targeted agents (e.g., PSC-Mac-CARs/CD 47 antibody) is less than or equal to 80%, less than or equal to 75%, less than or equal to 70%, less than or equal to 65%, less than or equal to 60%, less than or equal to 55%, less than or equal to 50%, less than or equal to 45%, less than or equal to 40%, less than or equal to 35%, less than or equal to 30%, is less than or equal to 25%, less than or equal to 20%, less than or equal to 15%, less than or equal to 10%, less than or equal to 5%, and less than or equal to 1%, relative to compositions comprising non-engineered immune cells and targeted agents (e.g., PSC-Mac/CD47 antibody).

### Dual Receptor AND-Gate Macrophages

In some embodiments, an engineered immune cell or population of engineered immune cells comprising a dual receptor sensing circuit (herein PSC-Mac-Dual). Without wishing to be bound by any particular theory, it is believed that dual sensing allows more precise and targeted killing of tumor cells and reduces the frequency and severity of undesired off-target effects. Such combinatorial approaches may circumvent the need for opsonin-mediated phagocytosis (e.g., the macrophage may be able to phagocytize the cell following binding two receptors).

In some embodiments, the engineered immune cells (e.g., PSC-Mac-Dual cells) comprise a first circuit configured to initiate a second circuit. In some cases, the first circuit comprises a first surface receptor capable of sensing and responding to an extracellular signal, such as, for example, a synthetic Notch receptor (herein synNotch). synNotch is known in the art to comprise an extracellular antigen recognition domain, a Notch core regulatory region, and an intracellular domain. Other synthetic pathways are also possible.

Non-limiting examples include chimeric antigen receptor, MESA, Tango, ChaCha, and GEMS.

In some embodiments, the extracellular antigen recognition domain comprises a single-chain variable fragment, scFv. The scFv may comprise a fusion protein or a chimeric protein. In some embodiments, the scFv comprises a chimeric protein made up of light land heavy chains of immunoglobins connected with a short linker peptide. Exemplary motifs that may be targeted by the scFv (e.g., antigens) include, but are not limited to, BCMA, CD19, CD22, CD20, CD138, CD33, CD123, PSMA, Igk, LeY, ROR1, biotin, CD171, EGFRVIII, FAP, FR, glypican-3, HER2, MUC-1, IL13Rα2, mesothelin, NKG2D, and PD1.

In some embodiments, the intracellular domain comprises a transcriptional factor configured to regulate expression of a target gene (e.g., it initiates the second circuit). Without wishing to be bound by any particular theory, it is believed that binding of the scFv to a target antigen stimulates a series of conformational changes in the Notch core regulatory region that releases the intracellular domain from the transmembrane domain. Once released, the intracellular domain initiates transcription of the target gene. In a preferred set of embodiments, the transcriptional domain is Gal4DBD-VP64, or TetR-VP64. Other transcriptional domains are also possible.

In some embodiments, the intracellular domain initiates transcription of a second circuit that results in the expression of a second surface receptor. In a preferred set of embodiments, the second circuit comprises the CAR-P constructs (e.g., such as those described herein) configured to initiate phagocytosis following binding of the CAR to its target antigen. As described elsewhere herein CAR-P constructs may be configured to bind to different antigens. Non-limiting examples include, but are not limited to, BCMA, CD19, CD22, CD20, CD138, CD33, CD123, PSMA, Igk, LeY, ROR1, biotin, CD171, EGFRVIII, FAP, FR, glypican-3, HER2, MUC-1, IL13Rα2, mesothelin, NKG2D, and PD1. Other secondary circuits are also possible.

Thus, in some embodiments, binding of synNotch on the surface of PSC-Mac-Dual cells to its target receptor on a target cell activates CAR-P transcription. Subsequent binding of the CAR-P receptor to its target receptor activates the macrophage to phagocytize the target cell. Alternatively, target cells expressing the synNotch antigen but lacking the CAR-P antigen will not be phagocytized by the engineered macrophages. Thus, both synNotch antigens and CAR-P antigens must be present on the target cell in order to initiate macrophage phagocytosis.

In some embodiments, the PSC-Mac-Dual cells do not exhibit ligand-independent activation (LIA) (e.g., to eliminate intracellular synNotch signaling in the absence of an extracellular binding event). For example, it is known in the art that cells comprising the synNotch construct may exhibit intracellular signaling (via synNotch) even if the extracellular antigen recognition domain has not bound its target antigen.

### Combinations

The engineered immune cells (e.g., macrophages) of the present disclosure may comprise any combination of elements described herein. For example, in some embodiments, the pharmaceutical composition comprises PSC-Macs comprising genomes that have been edited to knock-in and/or knock-out one or more genes of interest. For example, in some cases, the SIRPa gene is knocked-out, and a CAR-P gene construct is knocked-in (herein PSC-Mac-SIRPa-CAR)

In some embodiments, the combination comprises PSC-Mac-SIRPa-CAR cells and an antibody. The antibody may be a CD47 antibody and/or any subclass of IgG antibodies (e.g., IgG1, IgG2, IgG3, and IgG4) known to those of skill in the art.

In some embodiments, the engineered immune cells comprise PSC-Macs comprising genomes that have been edited to eliminate expression of SIRPa, and to allow expression of synNotch, which upon activation, is configured to control expression of an inducible CAR-P (herein PSC-Mac-SIRPa-DUAL). In some embodiments, activation of the CAR-P pathway via binding of the CAR-P to its target antigen, stimulates the immune cell to phagocytize the target cell.

In some embodiments, the engineered immune cells comprise a population of PSC-Mac-SIRPa-DUAL cells and an antibody. The antibody may be a CD47 antibody and/or any subclass of IgG antibodies (e.g., IgG1, IgG2, IgG3, and IgG4) known to those of skill in the art.

### Methods of Producing Engineered Immune Cells and Uses Thereof

Aspects of the present disclosure relate to methods of producing engineered immune cells (e.g., macrophages) with increased phagocytosis and/or enhanced tumor recognition capabilities. In some embodiments, the method comprises editing the genome of pluripotent stem cells (e.g., human pluripotent stem cells, PSC). In some cases, a target gene is knocked-in or knocked out prior to differentiation into a target immune cell (e.g., macrophages).

The PSC cell genome may be edited using any technique known to those of skill in the art. For example, in some embodiments, genes may be knocked-down (e.g., transient reduction in the expression of the gene of interest). Methods of knocking-down genes are known in the art and may target transcriptional and/or post-transcriptional processes. Non-limiting examples of methods used to knock-down genes of interest include, but are not limited to, genomic imprinting, paramutation, transposon silencing (or histone modifications), transgene silencing, position effect, RNA-directed DNA methylation, RNA interference, RNA silencing, and nonsense mediated decay. Other methods are also possible.

In some embodiments, the gene of interest may be knocked-out (e.g., target gene is made inoperative). Methods of knocking-out genes are known in the art and include, but are not limited to, homologous recombination and/or site-specific nucleases (e.g., zinc-fingers, TALENS, and/or CRISPR/Cas9).

In some embodiments, a CRISPR/Cas system is used to edit the genome. The CRISPR/Cas system may comprise one or more guide sequences (e.g., sgRNAs) designed to recognize target sequences (e.g., exon 1 of SIRPa). In various embodiments, the Cas system may be complexed, bound, or otherwise associated with (e.g., via any type of covalent or non-covalent bond) one or more guide sequences. The guide sequence becomes associated or bound to the Cas system and directs its localization to a specific target sequence having complementarity to the guide sequence or a portion thereof. The particular design embodiments of a guide sequence will depend upon the nucleotide sequence of a genomic target sequence (i.e., the desired site to be edited) and the type of Cas protein present in the system, among other factors, such as PAM sequence locations, percent G/C content in the target sequence, the degree of microhomology regions, secondary structures, etc.

In some embodiments, the Cas system is encoded in a vector and/or plasmid. Those of skill in the art will understand that introduction of extracellular DNA into cells may be accomplished via transfection. A number of transfection techniques are known in the art and may be used to transfect the cells of the current disclosure. Examples of transfection methods include, but are not limited to, electroporation, calcium phosphate exposure, and liposome-based transfections (e.g., Lipofectamine). Viral-based methods (e.g., transduction) are also possible and involve the use of a viral vector to carry specific nucleic acid sequence into the host cell. Non-limiting examples include retroviruses (e.g., lentiviruses), adenoviruses, adeno-associated viruses, and herpes viruses.

In some embodiments, the editing efficiency of the technique is between 50% and 100%. For example, in some cases, the editing efficiency of the technique is greater than or equal to 50%, greater than or equal to 60%, greater than or equal to 70%, greater than or equal to 80%, greater than or equal to 90%, greater than or equal to 95%, greater than or equal to 98%, greater than or equal to 99%, or greater than or equal to 100%. In other cases, the editing efficiency of the technique is less than or equal to 100%, less than or equal to 99%, less than or equal to 98%, less than or equal to 95%, less than or equal to 90%, less than or equal to 80%, less than or equal to 70%, less than or equal to 60%, and less than or equal to 50%.

### Engineered Immune Cells: SIRPa-Knockout

Aspects of making and using the engineered immune cells of the disclosure relate to editing the PSC genome to eliminate expression of a receptor. In certain embodiments, the receptor is SIRPa.

In a preferred set of embodiments, the method is a knockout method comprising a CRISPR/Cas system. In some embodiments the CRISPR/Cas system comprises Cas9, although other Cas equivalents may be used (e.g., Cas12e, Cas12d, Cas12a, Cas12b1, Cas13a, Cas12c, etc.). The CRISPR/Cas system may be used, for example, to cleave the genome at a desired location (e.g., AAVSI safe harbor site locus). In some embodiments, the Cas9 system expresses a marker protein, for example, to enable monitoring of expression levels (e.g., GFP). Any marker protein known to the skilled artisan may be used, including, but not limited to green fluorescent protein.

In some embodiments, the method comprises transfecting PSCs with one or more plasmids containing a Cas system targeting the 1^{st} and 3^{rd} exon of SIRPa (e.g., sgRNAs target the 1^{st} and 3^{rd} exons of SIRPa). Other exons may also be targeted. In some embodiments, the PSCs are transfected with more than one plasmid. For example, in some embodiments, the PSC cells are transfected with at least one plasmid, at least two plasmids, at least three plasmids, at least four plasmids, at least five plasmids, and at least six plasmids.

In some embodiments, the method comprises culturing the transfected PSCs for at least 24 hours. In some cases, the PSCs may be cultured for greater than or equal to 5 hours, greater than or equal to 10 hours, greater than or equal to 15 hours, greater than or equal to 20 hours, or greater than or equal to 24 hours after transfection. In some embodiments, the PSCs are cultured for less than or equal to 24 hours, less than or equal to 20 hours, less than or equal to 15 hours, less than or equal to 10 hours, or less than or equal to 5 hours after transfection.

In some embodiments, the cultured PSCs are sorted (e.g., via FACS) to isolate cells expressing the Cas expression system (e.g., cells expressing GFP following transfection with Cas9-GFP). Sorted cells, in some embodiments, are subsequently expanded and passaged to create clonal banks, which are subsequently analyzed for the presence of insertions/deletions (e.g., INDELs) containing disruptive deletions or introduction of stop codons that render the SIRPa gene inoperable.

In some embodiments, selected clones (e.g., clones with INDELs that inactivate the SIRPa gene) are differentiated into macrophages as described elsewhere herein, and the knockout efficiency (e.g., editing efficiency) determined, for example, using flow cytometry.

Other methods of knocking out a receptor (e.g., SIRPa) are possible. Non-limiting examples include homologous recombination and site-specific nucleases (e.g., zinc-fingers and TALENs). Other methods of knocking down a receptor are also possible and include, for example, siRNA, miRNA, shRNA, and RNAi.

### Engineered Immune Cells: Chimeric Antigen Receptor (CAR)

Several aspects of making and using the engineered immune cells (e.g., macrophages) of the disclosure relate to editing the PSC genome to induce expression of one or more receptors. In one set of embodiments, the genome is edited to induce expression of one or more chimeric antigen receptors (CARs) constructs. In some cases, the CAR construct is configured to stimulate phagocytosis upon binding to its target receptor.

In some embodiments, the CAR constructs are integrated into the PSC genome at a safe harbor site (SHS). SHS are genomic locations where new genes or genetic elements can be introduced without disrupting the expression or regulation of adjacent genes. In some embodiments, the SHS include the canonical human SAS, AAVS1, CCR5, CLYBL, and hROSA26. In a preferred set of embodiments, the CAR genes are inserted into the AAVS1 safer harbor locus.

The CAR gene can be inserted into the AAVSI safe harbor locus site using any technique known in the art. In a preferred set of embodiments, the CAR gene is inserted into the AAVSI using CRISPR/Cas9. Without wishing to be bound by any particular theory, this approach generally requires transfection with a first vector and a second vector. The first vector expresses the Cas9 endonuclease operatively linked to a first promoter (e.g., CMV, CAGGS, etc.) and an sgRNA targeting the AAVS1 locus under a second promoter (e.g., U6, etc.). This vector is responsible for creating the double strand DNA break at the desired location within the AAVSI locus. The gene of interest, located on the second vector, is then inserted into the genome using homology directed repair (HDR). The HDR pathway is a mechanism in cells that repairs DNA breaks using a DNA repair template that is homologous to the sites flanking upstream and downstream of the break site. Thus, in certain embodiments, the second vector comprises a gene of interest (e.g., the CAR construct) flanked by first homology arm (e.g., upstream) and a second homology arm (e.g., downstream) for the AAVS1 locus flanking the sgRNA targeting cut site.

In some embodiments, the first and/or second homology arms have about 900 bp homology with the AAVSI locus flanking the sgRNA targeting cut site. In some embodiments, the arms have greater than or equal to 400 bp homology, greater than or equal to 500 bp homology, greater than or equal to 600 bp homology, greater than or equal to 700 bp homology, greater than or equal to 800 bp homology, greater than or equal to 900 bp homology, and greater than or equal to 1000 bp homology with the AAVSI locus flanking the sgRNA targeting cut site. In some embodiments, the arms have less than or equal to 1000 bp homology, less than or equal to 900 bp homology, less than or equal to 800 bp homology, less than or equal to 700 bp homology, less than or equal to 600 bp homology, less than or equal to 500 bp homology, and less than or equal to 400 bp homology with the AAVSI locus flanking the sgRNA targeting cut site.

In some embodiments, the second vector further comprises a resistance marker. In a preferred set of embodiments, the resistance marker comprises puromycin, although any resistance marker known in the art may be used. In some embodiments, the gene of interest and/or the resistance marker may be operably linked to a promoter (e.g., an inducible promoter or a constitutively activated promoter.) Any inducible and/or constitutively activated promoter known in the art may be used, such as, for example, CMV or PGK, etc.

As described elsewhere herein, the CAR constructs of the present disclosure comprise an extracellular domain, a hinge sequence, a transmembrane domain, and an intracellular domain. Any extracellular domain, hinge sequence, transmembrane domain, and/or intracellular domain known in the art may be used. For example, extracellular domains configured to bind targets, such as, for example, BCMA, CD19, CD22, CD20, CD138, CD33, CD123, PSMA, Igk, LeY, ROR1, biotin, CD171, EGFRVIII, FAP, FR, glypican-3, HER2, MUC-1, IL13Rx2, mesothelin, NKG2D, and PD1 are contemplated.

In some embodiments the CAR constructs are configured to induce phagocytosis by stimulating one or more intracellular signaling pathways. Non-limiting examples of intracellular domains that trigger phagocytosis include Megf10, FcRγ, CD147, CD35, IgEγ rec (human), CD19 P13K do, CD284, 4-1BB, FCGR 1, FCGR2, and any combination thereof. In some embodiments, the intracellular signaling domain comprises two or more domains linked in tandem, for example, to increase the frequency of whole cell engulfment. In some embodiments, the tandem constructs comprise a FcRy domain fused CD19 cytoplasmic domain. For example, in some embodiments, the tandem construct comprises amino acids 500-534 Mouse CD19 (Uniprot CD19_MOUSE) fused to amino acids 19-86 of Mouse Fc ERG precursor (FCERG_MOUSE).

In some embodiments, cultured PSCs are transfected with the first and second vectors. Any method of introducing foreign DNA into a cell known in the art may be used, e.g., transduction. In a preferred set of embodiments, the cells are transfected with the vectors of interest using electroporation (e.g., using the 4D nucleofeactor by Lonza according to the manufacturer's instructions). In some cases, the molar ratio of the first plasmid to the second plasmid is varied. For example, in some embodiments, the molar ratio of the first: second plasmid is greater than or equal to 1:2, greater than or equal to 1:4, greater than or equal to 1:8, greater than or equal to 1:10, greater than or equal to 2:1, greater than or equal to 4:1, greater than or equal to 8:1, and greater than or equal to 10:1. In other cases, the molar ratio of the first: second plasmid is less than or equal to 10:1, less than or equal to 8:1, less than or equal to 4:1, less than or equal to 2:1, less than or equal to 1:10, less than or equal to 1:8, less than or equal to 1:4, and less than or equal to 1:2.

In some embodiments, the transfected PSC cells are cultured for between 5 days and 4 weeks in the presence of puromycin to select for cells with successful knock-in of the CAR-puromycin resistance cassette. For example, in some cases the cells are cultured for greater than or equal to 5 days, greater than or equal to 10 days, greater than or equal to 15 days, greater than or equal to 20 days, greater than or equal to 25 days, and greater than or equal to 30 days in the presence of puromycin. In some embodiments, the cells are cultured for less than or equal to 30 days, less than or equal to 25 days, less than or equal to 20 days, less than or equal to 15 days, less than or equal to 10 days, and less than or equal to 5 days.

In some embodiments, polymerase chain reaction genotyping, which is generally known in the art, is carried out to confirm knock-in of the CAR construct at the desired site (e.g., AAVSI site). In some embodiments, PSC cells comprising the CAR constructs may be subsequently differentiated into macrophages using the methods known in the art and as described herein.

### Synnotch Macrophages

In some embodiments, the engineered immune cells comprise a synthetic transmembrane notch signaling receptor, introduced at the pluripotent stem cell stage. In some embodiments, the synthetic receptor comprises of an extracellular scFv domain, a transmembrane domain, and an intracellular domain consisting of a cleavable notch element and synthetic transcription factor fusion. The synthetic receptor allows for cleavage of the intracellular portion of the notch core regulatory domain and release of the synthetic transcription factor upon binding of a fixed antigen recognized by the scFv fragment. In some embodiments, the engineered immune cells may comprise a transgene, which has its transcription regulated by promoter sequence targeted by the synthetic transcription factor. In some embodiments, the engineered immune cells are differentiated into phagocytotic cells (e.g., macrophages), such that when these phagocytotic cells (e.g., macrophages) encounter the scFv-targeting antigen, they respond by upregulating a synthetic gene of interest, introduced to promote phagocytosis, immune response, and chemotaxis to the region of interest.

### Derivation of Immune Cells From Human Pluripotent Stem Cells

Aspects of the present disclosure relate to differentiation of pluripotent stem cells into macrophages. The term “human pluripotent stem cells” as used herein refers to cells that are self-replicating and can be differentiated into cells and tissues of the three primary germ layers (e.g., ectoderm, mesoderm, and endoderm). The cells may be true embryonic stem cells (ES cells, e. g, are derived from human embryos or human fetal tissue), induced pluripotent stem cells (iPSC cells), by somatic cell nuclear transfer cells (ntES cells), or parthenogenetic embryonic stem cells (pES cells).

In some embodiments, differentiation of PSC into macrophages comprises culturing the cells in one or more cell culture mediums comprising one or more additives. In some embodiments, the one or more additives are present at a concentration of greater than or equal to 1 ng/ml, greater than or equal to 10 ng/mL, greater than or equal to 50 ng/ml, greater than or equal to 100 ng/mL, greater than or equal to 500 ng/ml, greater than or equal to 10 μg/mL, greater than or equal to 100 g/mL, greater than or equal to 500 μg/mL, greater than or equal to 1 mg/mL, greater than or equal to 10 mg/mL, greater than or equal to 20 mg/mL, greater than or equal to 50 mg/mL. In some embodiments, the one or more additives are present at a concentration of less than or equal to 50 mg/mL, less than or equal to 20 mg/mL, less than or equal to 10 mg/mL, less than or equal to 1 mg/mL, less than or equal to 500 μg/mL, less than or equal to 100 μg/mL, less than or equal to 10 g/mL, less than or equal to 500 ng/ml, less than or equal to 100 ng/ml, less than or equal to 50 ng/ml, less than or equal to 10 ng/ml and less than or equal to 1 ng/mL.

In some embodiments, the one or more additives are present at a concentration of greater than or equal 1 nM, greater than or equal to 10 nM, greater than or equal to 100 nM, greater than or equal to 500 nM, greater than or equal to 1 μM, greater than or equal to 10 μM, greater than or equal to 100 μM, greater than or equal to 500 μM, and greater than or equal to 1 mM. In some embodiments, the one or more additives are present at a concentration of less than or equal to 1 mM, less than or equal to 500 μM, less than or equal to 100 μM, less than or equal to 10 μM, less than or equal to 1 μM, less than or equal to 500 nM, less than or equal to 100 nM, less than or equal to 10 nM, and less than or equal to 1 nM.

In some embodiments, the PSCs of the current disclosure are derived from HI ES cells. Other cell sources may also be used (e.g., H9 embryonic stem cells, WIBR1, WIBR2, or WIBR3). In some embodiments, the pluripotent stem cells are cultured and maintained under feeder-free culture conditions. Any suitable culture media may be used to culture the ES cells (e.g., StemFlex™ from ThermoFisher Scientific). The culture media may comprise antibiotics (e.g., penicillin, streptomycin, etc.) and/or antifungal drugs. In some embodiments, the cell culture is incubated at a temperature of about 37° C. in a humidified incubator comprising about 5% CO₂ and 5% O₂.

In some embodiments, the cell culture media is transitioned to a medium specifically designed for culturing pluripotent stem cells (e.g., Essential 8, ThermoFisher Scientific). The timing of the transition is known in the art to be determined by the confluency, with approximately 70% confluency being the preferred cell density. In some embodiments, the cell culture media comprises Y27632 (e.g., ROCK inhibitor). In a preferred set of embodiments, the concentration of Y27632 is about 10 μm, although any suitable concentration may be used.

In some embodiments, the pluripotent stem cells are dissociated into a single cell suspension using a dissociation buffer (e.g., TrypLE Express, ThermoFisher Scientific). After dissociation, the ES cells are quantified, for example, using an automated cell counter (e.g., EVETM, NanoEntek) or a hemocytometer, and diluted. In some embodiments, the ES cells are diluted to a concentration of about 0.666×10⁶ cells/mL in embryoid body (EB) medium. Embryoid body medium comprises complete E8 medium supplemented with the following additives: penicillin, streptomycin, bone morphogenetic protein 4 (BMP), stem cell factor, vascular endothelial growth factor, and ROCK inhibitor.

In some embodiments, an aliquot of the dissociated cell suspension is plated into a well on a substrate (e.g., 96-well U-bottom ultra-low adherence well plates, Corning) and centrifuged. In a preferred set of embodiments, the cells are centrifuged at 200 RCF for 5 min, although other parameters may also be used. In some embodiments, additional EB medium (e.g., EB medium lacking ROCK inhibitor) may be added to each well, for example, one day after seeding of the cells in the wells of the substrate. In some cases, the EB cells may be transferred to a larger substrate (e.g., a 10 cm tissue culture-treated polystyrene dish) containing hematopoietic myeloid (HM) medium comprising X-VIVO 15 medium (Lonza) supplemented with the following additives: GlutaMax, beta-mercaptoethanol, macrophage colony-stimulating factor, and interleukin-3. In a preferred set of embodiments, the EB cells are provided fresh HM medium about every 4 days, although other feeding schedules are also possible.

In some embodiments, the cells are cultured for about 3 weeks. In some cases, the cells are cultured for greater than or equal to 1 week, greater than or equal to 2 weeks, greater than or equal to 3 weeks, and greater than or equal to 4 weeks. In some cases, the ES cells are cultured for less than or equal to 4 weeks, less than or equal to 3 weeks, less than or equal to 2 week, and less than or equal to 1 week.

EBs that float in culture are known in the art to consist, at least partially of, CD14 positive myeloid precursors (MPs) cells. In some embodiments, floating EB cells are collected and differentiated into macrophages by culturing them serum-free macrophage medium (SFMM). In some cases, the SFMM comprises glutamine containing no glucose RPMI medium supplemented with glucose, zinc sulfate heptahydrate, human transferrin, ethanolamine, sodium selenite, human insulin, minimal essential amino acids, sodium pyruvate (Gibco), penicillin, streptomycin, recombinant human albumin, and macrophage colony-stimulating factor. In a preferred set of embodiments, the MPs are differentiated into macrophages over about 10 days; although other times are also possible.

### Methods of Treatment

One aspect of the present disclosure relates to methods of treating a proliferative disease in a subject in need thereof. In certain embodiments, the proliferative disease is cancer. The methods include administering an engineered immune cell or a population of engineered immune cells (e.g., PSC-derived Macs).

In another aspect, the present disclosure provides methods of treating a cancer in a subject in need thereof, the methods comprising administering to the subject an effective amount (e.g., therapeutically effective amount) of (1) an engineered immune cell or population of engineered immune cells (e.g., PSC-derived Macs) and/or a targeted agent (e.g., an antibody) or (2) a pharmaceutical composition described herein. In certain embodiments, the engineered immune cell or population of engineered immune cells is a macrophage-directed immunotherapy. In some embodiments, the macrophage-directed immunotherapy and targeted agent are synergistic in treating the cancer, compared to the macrophage-directed immunotherapy and/or targeted agent alone.

In another aspect, the present disclosure provides methods of preventing a cancer in a subject in need thereof, the methods comprising administering to the subject an effective amount (e.g., prophylactically effective amount) of (1) a macrophage-directed immunotherapy and/or a targeted agent described herein, or (2) a pharmaceutical composition described herein. In certain embodiments, the macrophage-directed immunotherapy and/or targeted agent are synergistic in preventing the cancer, compared to the macrophage-directed immunotherapy and/or targeted agent alone.

In another aspect, the present disclosure provides methods of reducing, delaying, and/or preventing in a subject in need thereof the resistance of a cancer to a macrophage-directed immunotherapy and/or targeted agent, the methods comprising administering to the subject an effective amount of (1) a macrophage-directed immunotherapy and/or a targeted agent described herein, or (2) a pharmaceutical composition described herein. In certain embodiments, the macrophage-directed immunotherapy and targeted agent are synergistic in reducing, delaying, and/or preventing the resistance of the cancer to the macrophage-directed immunotherapy and/or targeted agent, compared to the macrophage-directed immunotherapy and/or targeted agent alone.

In certain embodiments, the macrophage-directed immunotherapy and/or targeted agent are administered to the subject at the same time. In certain embodiments, the macrophage-directed immunotherapy and/or targeted agent are administered to the subject at different times.

In another aspect, the present disclosure provides methods of inhibiting the proliferation of a cell, the methods comprising contacting the cell with an effective amount of (1) a macrophage-directed immunotherapy and/or a targeted agent described herein, or (2) a pharmaceutical composition described herein. In certain embodiments, the macrophage-directed immunotherapy and/or targeted agent are synergistic in inhibiting the proliferation of the cell, compared to the macrophage-directed immunotherapy and/or targeted agent alone.

In another aspect, the present disclosure provides methods of reducing, delaying, and/or preventing the resistance of a cell to a macrophage-directed immunotherapy and/or targeted agent, the methods comprising contacting the cell with an effective amount of (1) a macrophage-directed immunotherapy and/or a targeted agent described herein, or (2) a pharmaceutical composition described herein. In certain embodiments, the macrophage-directed immunotherapy and targeted agent are synergistic in reducing, delaying, and/or preventing the resistance of the cell to the macrophage-directed immunotherapy and/or targeted agent, compared to the macrophage-directed immunotherapy and/or targeted agent alone.

In another aspect, the present disclosure provides the macrophage-directed immunotherapies and/or targeted agents described herein for use in a method described herein (e.g., a method of treating cancer in a subject in need thereof, a method of preventing a cancer in a subject in need thereof, a method of reducing, delaying, and/or preventing in a subject in need thereof the resistance of a cancer to a macrophage-directed immunotherapy and/or targeted agent, a method of inhibiting the proliferation of a cell, or a method of reducing, delaying, and/or preventing the resistance of a cell to a macrophage-directed immunotherapy and/or targeted agent). In certain embodiments, the present disclosure provides the macrophage-directed immunotherapies and/or targeted agents for use in treating cancer in a subject in need thereof. In certain embodiments, the present disclosure provides a combination of the macrophage-directed immunotherapies and targeted agents for use in treating a cancer in a subject in need thereof.

In still another aspect, the present disclosure provides the pharmaceutical compositions described herein for use in a method described herein (e.g., a method of treating cancer in a subject in need thereof, a method of preventing a cancer in a subject in need thereof, a method of reducing, delaying, and/or preventing in a subject in need thereof the resistance of a cancer to a macrophage-directed immunotherapy and/or targeted agent, a method of inhibiting the proliferation of a cell, or a method of reducing, delaying, and/or preventing the resistance of a cell to a macrophage-directed immunotherapy and/or targeted agent). In certain embodiments, the present disclosure provides the pharmaceutical compositions for use in treating cancer in a subject in need thereof.

In certain embodiments, the methods described herein result in an increase in phagocytosis of cancer cells compared to treatment with the targeted agent alone. In certain embodiments, the methods described herein result in an increase in phagocytosis of cancer cells compared to treatment with the macrophage-directed immunotherapy alone. In certain embodiments, the methods described herein result in a synergistic increase in phagocytosis of cancer cells compared to treatment with the macrophage-directed immunotherapy and/or the targeted agent alone. In certain embodiments, the increase in phagocytosis of cancer cells is observed in a biological sample from a subject. In certain embodiments, the increase in phagocytosis of cancer cells is observed in an in vitro experiment.

In certain embodiments, the treatment results in an increase of at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% in phagocytosis of cancer cells compared to treatment with the macrophage-directed immunotherapy and/or the targeted agent alone. In certain embodiments, the treatment results of at least a 2-fold, at least a 3-fold, at least a 4-fold, at least a 5-fold, at least a 6-fold, at least a 7-fold, at least a 8-fold, at least a 9-fold, at least a 10-fold, at least a 20-fold, at least a 30-fold, at least a 40-fold, at least a 50-fold, at least a 60-fold, at least a 70-fold, at least a 80-fold, at least a 90-fold, at least a 100-fold, at least a 1000-fold, at least a 10000-fold, or at least a 100000-fold increase in phagocytosis of cancer cells compared to treatment with the macrophage-directed immunotherapy and/or the targeted agent alone. In certain embodiments, the cancer cells are lung cancer cells. In certain embodiments, the cancer cells are non-small cell lung cancer cells.

In certain embodiments, the macrophage-directed immunotherapies and targeted agents, or pharmaceutical compositions thereof, can be administered in combination with an anti-cancer therapy including, but not limited to, surgery, radiation therapy, transplantation (e.g., stem cell transplantation, bone marrow transplantation), and chemotherapy. In certain embodiments the macrophage-directed immunotherapies and targeted agents, or pharmaceutical compositions thereof, can be administered in combination with chemotherapy (i.e., one or more chemotherapeutic agents).

The methods described herein may be used to treat any cancer.

In certain embodiments, the cancer is a cancer that is commonly treated with chemotherapy. In certain embodiments, the cancer is a cancer that is commonly treated with immunotherapy. In some embodiments, the cancer is or comprises a solid tumor or hematological malignancy. In some embodiments, the cancer is or comprises a solid tumor. In some embodiments, the cancer is or comprises a hematological malignancy. In some embodiments, the cancer is a leukemia; a lymphoma; myelodysplasia; multiple myeloma; lung cancer (e.g., bronchogenic carcinoma, small cell lung cancer (SCLC), non-small cell lung cancer (NSCLC), adenocarcinoma of the lung); kidney cancer, acoustic neuroma; adenocarcinoma; adrenal gland cancer; anal cancer; angiosarcoma; appendix cancer; benign monoclonal gammopathy; biliary cancer, bladder cancer; breast cancer; brain cancer; bronchus cancer; carcinoid tumor; cervical cancer; choriocarcinoma; chordoma; craniopharyngioma; colorectal cancer; connective tissue cancer; epithelial carcinoma; ependymoma; endotheliosarcoma; endometrial cancer; esophageal cancer; Ewing's sarcoma; ocular cancer; familiar hypereosinophilia; gall bladder cancer; gastric cancer; gastrointestinal stromal tumor (GIST); germ cell cancer; head and neck cancer; heavy chain disease; leiomyosarcoma (LMS); mastocytosis; muscle cancer; myelodysplastic syndrome (MDS); mesothelioma; myeloproliferative disorder (MPD); neuroblastoma; neurofibroma; neuroendocrine cancer; osteosarcoma; ovarian cancer; papillary adenocarcinoma; pancreatic cancer; penile cancer; pinealoma; primitive neuroectodermal tumor (PNT); plasma cell neoplasia; paraneoplastic syndromes; intraepithelial neoplasms; prostate cancer; rectal cancer; rhabdomyosarcoma; salivary gland cancer; skin cancer; small bowel cancer; soft tissue sarcoma; sebaceous gland carcinoma; small intestine cancer, sweat gland carcinoma; synovioma; testicular cancer; thyroid cancer; urethral cancer; vaginal cancer; or vulvar cancer.

In certain embodiments, the cancer is bladder cancer, cervical cancer, dermatofibrosarcoma protuberans, endocrine tumors, neuroendocrine tumors, neuroblastoma, lung cancer (e.g., non-small cell lung cancer), anaplastic large cell lymphoma, glioblastoma multiforme, bile duct cancer, ovarian cancer, stomach cancer, colon cancer, rectal cancer, melanoma, colorectal cancer, brain cancer, head and neck cancer, thyroid cancer, soft tissue cancer, lung cancer, colon cancer, kidney cancer (e.g., papillary renal carcinoma), liver cancer, gastric cancer, gastrointestinal stromal tumor, giant cell tumor, esophageal cancer, gastroesophageal cancer, breast cancer, ovarian cancer, prostate cancer, endometrial cancer, pancreatic cancer, leukemia (e.g., acute myeloid leukemia), lymphoma, multiple myeloma, colon adenocarcinoma, lung adenocarcinoma, cutaneous melanoma, gastrointestinal cancer, anal cancer, glioblastoma, epithelian tumors of the head and neck, laryngeal cancer, oral cancer, myelodysplastic disorders, myeloproliferative disorders, ovarian epithelial cancer, fallopian tube cancer, primary peritoneal cancer, plexiform neurofibroma, skin cancer, soft tissue sarcoma, solid tumors with an NTRK gene fusion, or systemic mastocytosis.

In certain embodiments, the cancer is neuroblastoma, lung cancer (e.g., non-small cell lung cancer), anaplastic large cell lymphoma, glioblastoma multiforme, bile duct cancer, ovarian cancer, stomach cancer, colon cancer, rectal cancer, melanoma, colorectal cancer, brain cancer, head and neck cancer, thyroid cancer, soft tissue cancer, lung cancer, colon cancer, kidney cancer (e.g., papillary renal carcinoma), liver cancer, gastric cancer, gastroesophageal cancer, breast cancer, ovarian cancer, prostate cancer, endometrial carcinoma, pancreatic cancer, leukemia (e.g., acute myeloid leukemia), colon adenocarcinoma, lung adenocarcinoma, cutaneous melanoma, gastrointestinal cancer, anal cancer, glioblastoma, epithelian tumors of the head and neck, laryngeal cancer, and oral cancer. In certain embodiments, the cancer is lung cancer. In some embodiments, the cancer is non-small cell lung cancer (NSCLC).

### Pharmaceutical Compositions

Other aspects of the present disclosure relate to pharmaceutical compositions comprising any of engineered immune cells or combinations thereof, described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

In some embodiments, any of the engineered immune cells described herein are provided as part of a pharmaceutical composition. In some embodiments, the pharmaceutical composition comprises any of the engineered immune cells provided herein. In some embodiments, pharmaceutical composition comprises a polypeptide, a protein (e.g., an antibody), and a pharmaceutically acceptable excipient. Pharmaceutical compositions may optionally comprise one or more additional therapeutically active substances.

In some embodiments, compositions provided herein are formulated for delivery to a subject, for example, to a human subject, in order to deliver an engineered cell or population of engineered immune cells within the subject. In some embodiments, cells are obtained from the subject and contacted with any of the pharmaceutical compositions provided herein. In some embodiments, cells removed from a subject and contacted ex vivo with a pharmaceutical composition are re-introduced into the subject. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

The pluripotent stem cells, any their progeny, as described herein, may be allogeneic or autologous in various embodiments. In some embodiments somatic cells, e.g., fibroblasts, are obtained from a subject who is to be treated, and iPSCs are generated from said cells, which are then engineered and differentiated into macrophages and administered to said subject as described herein. In some embodiments somatic cells, e.g., fibroblasts, obtained from a healthy donor, who may or may not be genetically matched to the subject to be treated. iPSCs generated from such somatic cells are differentiated into macrophages and administered to the subject as described herein. In other embodiments, the pluripotent stem cells may be engineered to be HLA deficient, thus making them universal donor cells.

Formulations of the pharmaceutical compositions described herein may be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single-or multi-dose unit.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated in its entirety herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., to treat cancer. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials may be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition may be contained within a lipid particle or vesicle, such as a liposome or microcrystal, which is also suitable for parenteral administration. The particles may be of any suitable structure, such as unilamellar or plurilamellar, so long as compositions are contained therein. Compounds may be entrapped in “stabilized plasmid-lipid particles” (SPLP) containing the fusogenic lipid dioleoylphosphatidylethanolamine (DOPE), low levels (5-10 mol%) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N, N, N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition may be provided as a pharmaceutical kit comprising (a) a container containing a compound of the disclosure in lyophilized form and (b) a second container containing a pharmaceutically acceptable diluent (e.g., sterile water) for injection. The pharmaceutically acceptable diluent may be used for reconstitution or dilution of the lyophilized compound of the disclosure. Optionally associated with such container(s) may be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the disclosure. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1

Macrophages have shown promise as cancer immunotherapies in their ability to phagocytose solid tumor cells. This process is however limited by the inhibition of phagocytosis through the CD47-SIRPa signaling axis. Inhibition of this axis via CD47-opsonizing antibodies has been shown to increase phagocytosis of cancers with upregulated CD47 surface receptors. However, requisite use of these antibodies and reliance on primary sources of human macrophages are limiting. Presented herein is a technology to circumvent these challenges as well as provide new functionality to macrophages as immunotherapies using engineered human pluripotent stem cells. Functional macrophages have been derived with respect to phagocytosis of cancer cells and then their functionality has been enhanced by engineering the parent stem cells. The ability to both knock-in and knock-out genes into the parent stem cells, and successfully derive macrophages, has been demonstrated. These macrophages were then conferred enhanced functionality such as increased phagocytosis, phagocytosis without the need for CD47 antibodies, compatibility with use of other antibodies that confer cancer specific phagocytosis, enhanced phagocytosis to selective antigens with the introduction of chimeric antigen receptors (CARs), and antigen responsive changes in cytokine production/gene expression with use of synthetic notch CARs. In addition, libraries of genes for KO function and intracellular domains of CARs for enhancing the function of macrophages, have been developed. Lastly, these different engineering strategies can all be combined into combination technologies for further functionalization of macrophages as immunotherapies.

### Derivation of Macrophages From Human Pluripotent Stem Cells

Myeloid progenitor cells were derived from H1 embryonic stem cells (ESC) as previously described (Brownjohn et al., 2018). Briefly, HI ESCs were cultured and maintained in feeder-free culture conditions using StemFlex™ (ThermoFisher Scientific) pluripotent stem cell medium supplemented with 100U of penicillin and streptomycin (P/S) (Gibco), and Matrigel™ (Corning) coated tissue culture polystyrene. All cell culture was carried out at 37°C in a humidified incubator with 5% CO2 and 5% O2. At approximately 70% confluence, the medium was transitioned to Essential 8 (ThermoFisher Scientific) pluripotent stem cell medium supplemented with P/S and 10 μM Y-27632 (ROCK) inhibitor (Stem Cell Technologies) on day-1. On day 0, the HI ESCs were dissociated into a single cell suspension using TrypLE Express (ThermoFisher Scientific) and counted on an EVETM automated cell counter (NanoEntek). The HI ESCs were diluted to 0.666×10⁶ per mL in embryoid body (EB) medium consisting of complete E8 medium with P/S supplemented 50 ng/mL bone morphogenetic protein 4 (Peprotech), 20 mg/mL stem cell factor (Peprotech), and 50 ng/ml vascular endothelial growth factor, with 10 μM ROCK inhibitor. 150 μL of this cell suspension per well was plated into a 96-well U-bottom ultra-low adherence well plates (Corning) and centrifuged at 200 RCF for 5 min. Care was taken to minimize pipetting of the ESCs to promote the best EB formation. On day 2, 150 μL of EB medium without ROCK inhibitor was added to each well. On day 4, the EBs were transferred to a 10 cm tissue culture-treated polystyrene dish in 12 mL of hematopoetic myeloid (HM) medium consisting of X-VIVO 15 medium (Lonza) supplemented with 2 mM GlutaMax (Gibco), 55 μM beta-mercaptoethanol (Sigma Aldrich), 100 ng/ml macrophage colony-stimulating factor (Peprotech), and 25 ng/ml interleukin-3 (Peprotech). The plated EBs were fed on a schedule consisting of the addition of 12 mL fresh HM medium on Mondays and the exchange of medium for 12 mL of HM medium on Fridays. Care was taken not to lose any EBs that began to float. Around 2-3 weeks after EB plating, floating CD 14+ myeloid precursors (MPs) began to appear and were collected on Fridays while straining and returning any floating EBs to the HM media culture. The collected MPs were differentiated in macrophages by plating the cells in a 10 cm tissue culture-treated polystyrene dish in 12 mL serum-free macrophage medium (SFMM) consisting of glutamine containing no glucose RPMI medium (Gibco) supplemented with 1 mM glucose (Sigma Aldrich), 0.432 mg/L zinc sulfate heptahydrate (Sigma Aldrich), 7.5 mg/L human transferrin (holo) (Sigma Aldrich), 1.9 mg/L ethanolamine (Sigma Aldrich), 0.005 mg/L sodium selenite (Sigma Aldrich), 1.7 nM human insulin (Sigma Aldrich), 1:100 dilution of minimal essential amino acids (Gibco), 1:100 dilution of sodium pyruvate (Gibco), P/S, 0.2 g/L of recombinant human albumin (CellaStim), and 100 ng/ml macrophage colony-stimulating factor. The MPs were differentiated into macrophages over 10 days in SFMM with the same feeding schedule as the EBs in HM medium.

### SirpA KO Methodology:

Three sgRNAs were designed, targeting th^{e} 1st and 3rd exons of SirpA using ChopChop software. Oligos representing these guides were cloned into a Cas9-EGFP expressing plasmid with the gRNA scaffold driven by the hU6 promoter using restriction enzyme and ligase reactions. A mixture of the three plasmids containing the three sgRNA sequences was electroporated into 1 million human pluripotent stem cells and the cells were plated for 24 hours. After 24 hours, the PSCs were sorted into 96-well plates FACs and selected for GFP-positive single cells. These single-cell clones were expanded and passaged to create clonal banks and to extract genomic DNA. Using the clonal genomic DNA, PCR was performed on the targeted exons and the sequences were analyzed for INDELs containing disruptive deletions or introductions of stop codons to select putative clones. Candidate clones were then differentiated into macrophages and KO of SirpA was screened using flow cytometry for KO efficacy using the cancer cell co-culture with cetuximab.

### Co-Culture Experiments

A range of 5, 10, and 20 k wild-type stem cell-derived macrophages was used for 25 or 50 k cancer cells in a 384 well plate. For CAR and SirpA KO cells, 10 k macrophages were used. The cells were observed for eight days and imaged every 4 hours. CAR design, editing, and experiments

hESC cells were electroporated with a plasmid containing an AAVSI-targeting sgRNA and Cas9-2A-GFP (Addgene Plasmid #38138) and a second donor template plasmid containing a splice acceptor with puromycin for selection from the endogenous AAVS1 expression, a constitutively expressed CAR construct using the CAGGS promoter, and 800-900 bp homology arms for the AAVSI locus flanking the sgRNA targeting cut site. These intracellular domains were previously described in Morrissey et al .. Electroporations were done using a 4D nucleofector (Lonza) according to the manufacturer's instructions and 1:4 molar ratio of Cas9 to donor template plasmid. After electroporation, cells were plated in a 10 cm Matrigel-coated dish and allowed to recover for 3 days. After recovery, cells were selected with puromycin from the endogenous AAVSI expression for 5 days at 1 μg/mL.

The CAR design consisted of the following fragments in N to C terminus order, a conserved CAR domain of: CD28 signal peptide, CD19 scFV VL chain, a linker chain, CD19 scFV VH chain, CD8a hinge domain, and a CD28 transmembrane domain. Data included in the disclosure included intracellular stimulation domains of ) Cytosolic sequence:
aa19-86 Mouse Fc ERG precursor (UniprotP20491 (FCERG_MOUSE) as CAR-GFP and Cytosolic sequence: aa 500-534 Mouse CD19 (Uniprot CD19_MOUSE) fused to aa 19-86Mouse Fc ERG precursor (FCERG_MOUSE) as CAR-Tandem. For co-coculture experiments, WT, CAR-GFP, and CAR-Tandem PSC-derived macrophages were cultured for 7 days with CD 19+ or CD19-tdTomato DLD-1 adenocarcinoma lines. Increased phagocytosis was observed of the both CAR lines against CD19-DLD-1 cells and a greater response to CD19+ cells was also observed.

### Example 2

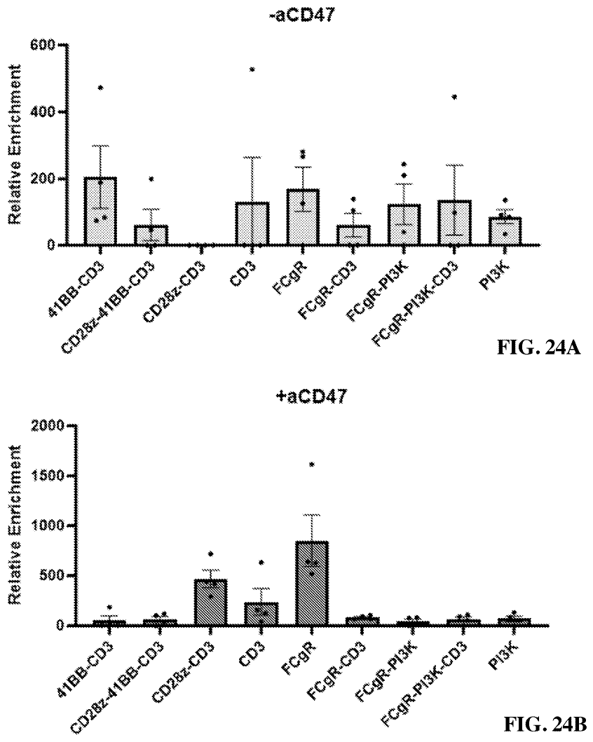

Pooled macrophages with CARs containing a common extracellular anti-CD19 scFv domain and varied intracellular signaling domains (ISD) were mixed with fluorescently labeled CD19-expressing DLD-1 colorectal adenocarcinoma cells with (FIG. 24B) and without CD47 (FIG. 24A) opsonizing antibodies. Fluorescence-activated cell sorting was used to isolate highly phagocytic cells after 3 hours by sorting the cells positive for the DLDI and orthogonal macrophage fluorescent labels. DNA was extracted from these sorted highly phagocytic cells, and the variable ISD domain of the chimeric antigen receptor was amplified using PCR. The PCR products were sequenced using long-read nanopore sequencing and then the read frequency of the total sampled was determined via mapping back to the known CAR sequences. An increase in relative enrichment with CD47 opsonizing antibody reveals that a single, human epsilon subunit of the Fc gamma receptor 1 promotes the highest level antigen-mediated phagocytosis in stem cell-derived CAR macrophages.

### REFERENCES

Brownjohn, P. W. et al. Functional Studies of Missense TREM2 Mutations in Human Stem Cell-Derived Microglia. Stem Cell Reports 10, 1294-1307 (2018).

Morrissey M A, Williamson A P, Steinbach A M, Roberts E W, Kern N, Headley M B, Vale R D. Chimeric antigen receptors that trigger phagocytosis. Elife. 2018 June 4;7: e36688. doi: 10.7554/eLife.36688. PMID: 29862966; PMCID: PMC6008046.

### Equivalents And Scope

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. An engineered immune cell, comprising:
a genome that has been edited to eliminate expression of SIRPa in the cells;
wherein the engineered immune cell is an immune cell derived from a pluripotent stem cell or a primary cell, or a non-stem cell derived immune cell.

2. The engineered immune cell of claim 1, further comprising an IgG antibody, an antibody derivative, and/or fragment thereof configured to bind to an Fc receptor on the surface of the engineered immune cell.

3. The engineered immune cell of claim 2, wherein the IgG antibody, an antibody derivative, and/or fragment thereof comprises an antigen binding site configured to bind to an antigen on a target cell.

4. The engineered immune cell of claims 2 or 3, wherein the IgG antibody an antibody derivative, and/or fragment thereof is selected from the group consisting of IgG1, IgG2, IgG3, and IgG4.

5. The engineered immune cell of claim 1, wherein the pluripotent stem cell is selected from the group consisting of induced pluripotent stem cells (iPSC cells), true embryonic stem cells (ES cells), somatic cell nuclear transfer cells (ntES cells), and parthenogenetic embryonic stem cells (pES cells).

6. The engineered immune cell of any one of claims 1-5, wherein the pluripotent stem cell expresses the pluripotency factors NANOG, OCT4, and SOX2.

7. The engineered immune cell of any one of claims 1-6, wherein the pluripotent stem cell expresses an endoderm specific marker.

8. The engineered immune cell of claim 7, wherein the endoderm specific marker is GATA4 and SOX17.

9. The engineered immune cell of any one of claims 1-8, wherein the pluripotent stem cell expresses a mesoderm specific marker.

10. The engineered immune cell of claim 9, wherein the mesoderm specific marker is BRACHURY and EOMES.

11. The engineered immune cell of any one of claims 1-10, wherein the pluripotent stem cell expresses a neuroectoderm specific marker.

12. The engineered immune cell of any one of claims 11, wherein the neuroectoderm specific marker is NESTIN and SOX1/PAX6.

13. The engineered immune cell of any one of claims 1-12, wherein the engineered immune cell is selected from the group consisting of macrophages, neutrophils, monocytes, dendritic cells, eosinophils, T cells, NK cells, B cells, basophils, and mast cells.

14. The engineered immune cell of any one of claims 1-13, wherein the engineered immune cell is a macrophage.

15. The engineered cell of any one of claims 1-14, wherein the genome has been edited to reduce expression of SIRPa by between 50% and 90%.

16. An engineered immune cell, comprising:
a genome that has been edited to express one or more chimeric antigen receptors configured to bind to one or more target antigens on a cancer cell; and
wherein the genome is edited at a safe harbor locus site, wherein binding of the chimeric antigen receptor to the target antigen stimulates the engineered cell to phagocytize the cancer cell, and wherein the engineered immune cell is derived from a pluripotent stem cell or a primary cell, or a non-stem cell derived immune cell.

17. The engineered immune cell of claim 16, wherein the chimeric antigen receptor comprises an extracellular single-chain antibody variable fragment (scFv) configured to bind the target antigen, a hinge sequence, a transmembrane CD8 domain, and an intracellular domain.

18. The engineered immune cell of claim 16, wherein binding of the scFv domain to the target antigen activates a signaling cascade within the intracellular domain.

19. The engineered immune cell of claim 16, wherein the signaling cascade signals the engineered immune cell to phagocytize the cancer cell.

20. The engineered immune cell of claim 16-19, wherein the intracellular domain is selected from the group consisting of Megf10, FcRy, and CD19 PI3K domain.

21. The engineered immune cell of claim 16-20, wherein the pluripotent stem cell is selected from the group consisting of induced pluripotent stem cells (iPSC cells), true embryonic stem cells (ES cells), somatic cell nuclear transfer cells (ntES cells), and parthenogenetic embryonic stem cells (pES cells).

22. The engineered immune cell of claim 16-21, wherein the pluripotent stem cells express NANOG, OCT4, and SOX2.

23. The engineered immune cell of claim 16-22, wherein the pluripotent stem cells express GATA4, SOX17, BRACHURY, EOMES, NESTIN, and SOX1/PAX6.

24. The engineered immune cell of claim 16-23, wherein the engineered immune cell is selected from the group consisting of macrophages, neutrophils, monocytes, dendritic cells, eosinophils, T cells, NK cells, B cells, basophils, and mast cells.

25. The engineered cell of claim 16-24, wherein the engineered immune cell is a macrophage.

26. The pharmaceutical composition of claim 16-25, wherein the target antigen is CD19.

27. An engineered immune cell, comprising a genome that has been edited to:
(a) eliminate expression of SIRPa in the engineered immune cells or population of engineered immune cells; and
(b) to allow expression of one or more chimeric antigen receptors configured to bind to one or more target antigens on a target cell;
wherein binding of the one or more chimeric antigen receptors to the one or more target antigens stimulates the engineered cell to phagocytize the target cell.

28. The engineered immune cell of claim 27, further comprising an IgG antibody an antibody derivative, and/or fragment thereof configured to bind to an Fc receptor on the surface of the immune cell.

29. The engineered immune cell of claim 28, wherein the IgG antibody an antibody derivative, and/or fragment thereof comprises an antigen binding site configured to bind to an antigen on the target cell.

30. The engineered immune cell of claims 27-29, wherein the chimeric antigen receptor comprises an extracellular single-chain antibody variable fragment (scFv) configured to bind a target antigen, a hinge sequence, a transmembrane CD8 domain, and an intracellular domain.

31. The engineered immune cell of any one of claims 27-30, wherein the chimeric antigen receptor is incorporated into the genome at an AAVSI safe harbor locus site.

32. The engineered immune cell of claims 27-31, wherein the engineered immune cell is derived from a pluripotent stem cell.

33. The engineered immune cell of claim 32, wherein the pluripotent stem cell is selected from the group consisting of induced pluripotent stem cells (iPSC cells), true embryonic stem cells (ES cells), somatic cell nuclear transfer cells (ntES cells), and parthenogenetic embryonic stem cells (pES cells).

34. The engineered immune cell of claim 27-33, wherein the engineered immune cell is derived from a primary cell or a non-stem cell derived immune cell.

35. An engineered immune cell or population of engineered immune cells, comprising:
at least one synthetic Notch receptors comprising an extracellular recognition domain configured to bind to at least one first antigens; and
at least one inducible chimeric antigen receptor (CAR) configured to bind to at least one second antigen
wherein binding of the at least one synthetic Notch receptor to the at least one first antigen drives expression of the at least one inducible chimeric antigen receptor, and
wherein the simultaneous binding of the at least one inducible chimeric antigen receptor to the at least one second antigen stimulates immune cell activation.

36. The engineered immune cell of claim 35, wherein the at least first antigen comprises a known cancer antigen.

37. The engineered immune cell of claim 35 or 36, wherein the at least second antigen comprises a known cancer antigen.

38. The engineered immune cell of any one of claims 35-37, wherein the at least one inducible chimeric antigen receptor comprises an extracellular single-chain antibody variable fragment (scFv), a transmembrane CD8 domain, and an intracellular signaling domain.

39. The engineered immune cell of claim 38, wherein the intracellular signaling domain comprises CD19.

40. The engineered immune cell of claim 39, wherein the extracellular single-chain antibody variable fragment is configured to bind to CD19.

41. The engineered immune cell of any one of claims 35-40, wherein the engineered immune cell is a macrophage.

42. The engineered immune cell of any one of claims 35-41, wherein the chimeric antigen receptor is incorporated into the genome at a safe harbor locus site.

43. The engineered immune cell of any one of claims 35-42, wherein the synNotch receptor is incorporated into the genome at a safe harbor locus site.

44. The engineered immune cell of claims 42 or 43, wherein the safe harbor locus site is selected from the group consisting of canonical human SAS, AAVSI, CCR5, and hROSA26.

45. The engineered immune cell of any one of claims 35-44, wherein the safe harbor locus site is AAVS1.

46. The engineered immune cell of any one of claims 35-45, wherein the engineered immune cell is derived from a pluripotent stem cell, a primary cell, or a non-stem cell-derived immune cell.

47. An engineered immune cell, comprising:
a genome that has been edited to:
(a) eliminate expression of SIRPa in the cell or population of cells;
(b) allow expression of a synthetic Notch receptor comprising an extracellular recognition domain configured to bind to a first antigen; a©(c) allow expression of an inducible chimeric antigen receptor configured to bind to second antigen,
wherein binding of the synthetic Notch receptor to the first antigen drives expression of the inducible chimeric antigen receptors, and
wherein binding of the inducible chimeric antigen receptor to the second antigen stimulates immune cell activation.

48. The engineered immune cell of claim 47, further comprising an IgG antibody an antibody derivative, and/or fragment thereof configured to bind to an Fc receptor on the surface of the engineered immune cell.

49. The engineered immune cell of claim 47 or 48, wherein the IgG antibody an antibody derivative, and/or fragment thereof comprises an antigen-binding site configured to bind to an antigen on a target cell.

50. The engineered immune cell of claim 47-49, wherein the engineered immune cell is a macrophage.

51. The engineered immune cell of claims 47-50, wherein the engineered immune cell is a derived from a pluripotent stem cell, a primary cell, or a non-stem cell derived immune cell.

52. The engineered immune cell of any one of claims 47-51, wherein the chimeric antigen receptor is incorporated into the genome at a safe harbor locus site.

53. The engineered immune cell of any one of claims 47-52, wherein the synNotch receptor is incorporated into the genome at a safe harbor locus site.

54. The engineered immune cell of claims 52 or 53, wherein the safe harbor locus site is selected from the group consisting of canonical human SAS, AAVS1, CCR5, and hROSA26.

55. The engineered immune cell of any one of claims 52-54, wherein the safe harbor locus site is AAVS1.

56. An engineered immune cell, comprising:
a genome that has been edited to eliminate expression of one or more target genes;
wherein the engineered immune cell is an immune cell derived from a pluripotent stem cell or population of stem cells, or primary cell, or a non-stem cell based immune cell.

57. The engineered immune cell of claim 56, wherein the one or more target genes is a macrophage inhibitory receptor.

58. The engineered immune cell of claim 57, wherein the macrophage inhibitory receptor is selected from the group consisting of LILRB1, LILRB2, LILRB3, LILRB4, and RGMB.

59. The engineered immune cell of claim 56-58, wherein the genome is further edited to express one or more chimeric antigen receptors configured to bind to one or more target antigens on a target cell;

60. The engineered immune cell of claim 59, wherein binding of the one or more chimeric antigen receptors to the one or more target antigens stimulates the engineered cell to perform a function.

61. The engineered immune cell of claim 60, wherein the function comprises phagocytizing a cell comprising the target antigen.

62. The engineered immune cell of claim 60 or 61, wherein the function comprises secreting one or more cytokines configured to activate a macrophage.

63. An engineered immune cell or population of engineered immune cells, comprising:
at least one synthetic Notch receptor comprising an extracellular recognition domain configured to bind to at least one first antigen; and
at least one inducible chimeric antigen receptor (CAR) configured to bind to at least one second antigen
wherein binding of the at least one synthetic Notch receptor to the at least one first antigen drives expression of the at least one inducible chimeric antigen receptor, and
wherein the simultaneous binding of the at least one inducible chimeric antigen receptor to the at least one second antigen stimulates secretion of one or more cytokines.

64. The engineered immune cell or population of engineered immune cells of claim 63, wherein the one or more cytokines are selected from the group consisting of INFα, INFβ INFγ, INFO, and IL-10.

65. The engineered immune cell or population of engineered immune cells of claims 63 or
64, further comprising a genome that has been edited to eliminate expression of a target gene.

66. A pharmaceutical composition comprising an antibody, an antibody, derivative, and/or fragment thereof and one or more of the engineered immune cells of claims 1-65.

67. The pharmaceutical composition of claim 66, wherein the antibody, antibody derivative, and/or fragment thereof is selected from the group consisting of IgG, IgM, IgE, and IgA.

68. The pharmaceutical composition of claim 66 or 67, wherein the antibody, antibody derivative, and/or fragment thereof is an IgG antibody.

69. The pharmaceutical composition of claims 66-68, wherein the antibody is a CD47 antibody.

70. A pharmaceutical composition comprising an IgG antibody, an IgG antibody, derivative, and/or fragment thereof and one or more of the engineered immune cells of claims 1-65.

71. A pharmaceutical composition comprising a CD47 antibody, a CD47 antibody derivative, and/or fragment thereof and one or more of the engineered immune cells of claims 1-65.

72. A pharmaceutical composition comprising a CD47 antibody, a CD47 antibody derivative, and/or fragment thereof, an IgG antibody, an IgG antibody derivative, and/or fragment thereof and one or more of the engineered immune cells of claims 1-65.

73. The pharmaceutical composition of any one of claims 66-72, further comprising one or more excipients

74. The pharmaceutical composition of any one of claims 66-73, further comprising one or more adjuvants.

75. The pharmaceutical composition of claim 74, wherein the adjuvant comprises a chemotherapeutic agent.

76. The pharmaceutical composition of claim 74 or 75 wherein the adjuvant comprises an immunotherapy.

77. The pharmaceutical composition of claim 76, wherein the immunotherapy is selected from the group consisting of immune checkpoint inhibitors, T-cell transfer therapy, monoclonal antibodies, vaccines, and cytokines.

78. A method of treating cancer in a subject in need thereof, the method comprising administering one or more engineered immune cell claims 1-65.

79. A method of treating cancer in a subject in need thereof, the method comprising administering one or more pharmaceutical composition of claims 66-77.

80. The method of claims 78 and 79, further comprising administering an additional medical therapy selected from the group consisting of chemotherapy, radiation therapy, surgery, immunotherapy, targeted therapy, stem cell transplantation therapy, and hormone therapy.

81. The method of any one of claims 78-80, further comprising administration of one or more immunosuppressant drugs.

82. A method of enhancing cancer cell phagocytosis in a subject in need thereof, the method comprising administering one or more of the engineered immune cell of claims 1-65.

83. A method of enhancing cancer cell phagocytosis in a subject in need thereof, the method comprising administering one or more pharmaceutical composition of claims 66-77.

84. The method of enhancing cancer cell phagocytosis of claims 82 and 83, further comprising administering one or more medical treatments.

85. The method of enhancing cancer cell phagocytosis of claim 84, the one or more medical treatments are selected from the group consisting of chemotherapy, radiation therapy, surgery, immunotherapy, targeted therapy, stem cell transplantation therapy, and hormone therapy.

86. A method of engineering an immune cell with increased anti-tumor activity, the method comprising:
(a) obtaining a plurality of pluripotent stem cells and differentiating said stem cells into CD45+/CD34+ myeloid progenitor cells;
(b) editing the genome of the CD45+/CD34+ myeloid progenitor cells to include a chimeric antigen receptor gene and/or a synNotch gene inside a locus safe harbor locus site;
(c) differentiating the CD45+/CD34+ myeloid progenitor cells into CD14+ macrophages; and
(d) increasing the number of edited CD14+ macrophages in a bioreactor by at least 10-fold.

87. The method of claim 86, further comprising editing the genome to knockout expression of SIRPa.

88. The method of claim 86 or 87, wherein the plurality of pluripotent stem cells is selected from the group consisting of induced pluripotent stem cells (iPSC cells), true embryonic stem cells (ES cells), somatic cell nuclear transfer cells (ntES cells), and parthenogenetic embryonic stem cells (pES cells).

89. The method of any one of claims 86-88, wherein editing the genome utilizes a CRISPR/Cas system

90. The method of any one of claims 86-89, wherein the safe harbor locus site is selected from the group consisting of canonical human SAS, AAVSI, CCR5, and hROSA26.

91. The method any one of claims 86-90, wherein the safe harbor locus site is AAVSI.

92. The method any one of claims 86-91, wherein editing the genome does not involve a lentivirus.

93. A method for screening for engineered immune cells with enhanced phagocytotic activity, the method comprising:
(a) inducing gene knockout after differentiating PSC into macrophages using sgRNA libraries;
(b) enriching for phagocytic cells using fluorescence activated cell sorting;
(c) identifying the gene knocked out using traditional sequencing techniques; and
(d) creating a plurality of engineered immune cells, wherein the genome of the engineered immune cell has an edited genome.

94. The method of claim 93, wherein the method comprises a Xon system.

95. The method of claim 93 or 94, wherein the method comprises a degron inducible system.

96. The method of any one of claims 93-95, wherein the method comprises a tetracyline responsive silencer-tet 1 demethylation fusion protein.

97. The method of any one of claims 93-96, wherein the genome is edited at the AAVS1 locus.

98. An engineered immune cell, comprising:
a genome that has been edited to disrupt the ability of a target gene to function;
wherein the engineered immune cell is an immune cell derived from a pluripotent stem cell or a primary cell, or a non-stem cell derived immune cell.

99. The engineered immune cell of claim 98, wherein the genome is further edited to express one or more chimeric antigen receptors.

100. The engineered immune cell of claim 98 or 99, wherein the genome is further edited to comprise a synNotch receptor and at least one inducible chimeric antigen receptor.

101. The engineered immune cell of any one of claims 98-100, wherein the target gene is truncated during editing.
